(19) United States
(12) Patent Application Publication
(10) Pub. No.: US 2009/0298139 A1
(43) Pub. Date: Dec. 3, 2009

(54) Method for Polymer Synthesis Using Microfluidic Enzymatic Cascade

(76) Inventors: Jin Zou, Lexington, MA (US); David L. Kaplan, Concord, MA (US); Jin Zou, Woburn, MA (US); Peter Y. Wong, Brighton, MA (US); David L. Kaplan, Concord, MA (US)

Correspondence Address: MEDLEN & CARROLL, LLP, 101 HOWARD STREET, SUITE 350, SAN FRANCISCO, CA, 94105, US

(21) Appl. No.: 12/224,149

(22) PCT Filed: Feb. 27, 2007
(86) PCT No.: PCT/US2007/005107

§ 371 Date: Mar. 20, 2009

Related U.S. Application Data
(60) Provisional application No. 60/777,735, filed on Feb. 28, 2006.

(51) Int. Cl. C12P 17/16; B01J 19/00

(52) U.S. Cl. 435/118; 435/289.1; 422/131

(57) ABSTRACT

The present invention discloses a method for producing polymers in a microscale device. The system utilizes a symmetrically branched system of microchannels interconnecting a plurality of loading decks and re-action chambers. The fluid flow is manipulated by the placement of capillary check valves, mixing areas, and microcomb filters. The system provides for cascading enzymatic biosynthesis pathways wherein any variety of enzymes and reactants can be introduced into the system to produce a final product.

### FIELD OF INVENTION

The present invention is related to general enzymatic reactions, especially those involving the synthesis of new polymers or breakdown of polymers to create monomers. This system and technology can be broadly used in various areas of polymer synthesis research, microfluidic engineering, elucidating metabolic relationships among pathways related to metabolic engineering and pharmaceuticals development. In one embodiment, a microfluidic device produces a polymer by enzymatic cascade.

### BACKGROUND

Integrating an entire conventional, general-purpose chemistry laboratory onto a single microchip is many years away. Verpoorte et al., “Microfluidics meets MEMS” IEEE, 91(6) (2003). Even the scaling down of an entire multi-step enzymatic cascade synthesis onto a small chip remains a significant challenge for microsystem design and integration. In addition, enzyme activity temperature, purification, and strict sequential action by enzymatic catalytic reactions make both macro- and microsynthesis a difficult one in the biochemical domain.

On the other hand, a simple and reliable design, with monolithic fluidic manipulating functionality requires a breakthrough design in microfluidic domain with a deep understanding of mechanical engineering. ‘Microscale enzymatic polymerization on a chip’ thus remains as challenge that crosses biochemical and mechanical engineering. Moreover, the manufacturability of this design needs to be guaranteed at the micron level, while still achieving low cost.

Clearly, what is needed in the art is a device and method designed specifically for microfluidic enzyme cascades that produce specific compounds quickly, efficiently, and at a minimum cost.

### SUMMARY OF THE INVENTION

The present invention is related to general enzymatic reactions, especially those involving the synthesis of new polymers or breakdown of polymers to create monomers. This system and technology can be broadly used in various areas of polymer synthesis research, microfluidic engineering, elucidating metabolic relationships among pathways related to metabolic engineering and pharmaceuticals development. In one embodiment, a microfluidic device produces a polymer by enzymatic cascade.

In one embodiment, the present invention contemplates a method, comprising: a) providing: i) a substrate loading deck into which a sample comprising a first reactant is introduced; ii) a plurality of reactant loading decks into which at least one additional reactants are introduced; iii) a plurality of reaction chambers comprising at least one enzyme, wherein said chambers are in fluidic communication with said substrate loading deck and said reactant loading decks; b) introducing said substrate into said substrate loading deck under conditions such that said substrate moves into a first reaction chamber; c) introducing an additional reactant into a first reactant loading deck under conditions such that said second reactant moves into said first reaction chamber; and d) reacting said sample and said additional reactant in said first reaction chamber under conditions such that a polymer is formed. In one embodiment, the reaction chambers further comprise a microcomb filter for separating said polymer. In one embodiment, the method further comprises symmetrically branched microchannels fluidly connecting said sample loading deck, said reactant loading decks reaction chambers. In one embodiment, the reaction chamber further comprises at least one side channel for collecting unreacted sample and unreacted additional reactants. In one embodiment, the introducing comprises an injection. In one embodiment, the microcomb filter comprises two side microcombs. In one embodiment, the microcomb filter comprises a central microcomb and two side microcombs. In one embodiment, the substrate comprises an antioxidant. In one embodiment, the additional reactant comprises 2,2,2-trifluoroethyl methacrylate. In one embodiment, the enzyme comprises a lipase.

In one embodiment, the present invention contemplates a system, comprising: a) at least one substrate loading deck for introducing a substrate into a first microchannel; b) a plurality of reactant loading decks into which at least one reactant is introduced into a second microchannel; c) a mixing area wherein said substrate from said first microchannel and said reactant from said second microchannel intersect, thereby forming a first reaction mixture in a third microchannel;

d) a first reaction chamber comprising a first enzyme in fluidic communication with said third microchannel wherein said first reaction mixture forms a second reaction mixture; and e) a second reaction chamber comprising a second enzyme in fluidic communication with said first reaction chamber wherein said second reaction mixture forms a polymer. In one embodiment, the reaction chamber further comprises a microcomb filter for separating said polymer. In one embodiment, the reaction chamber further comprises at least one cross channel for collecting unreacted sample and unreacted reactant. In one embodiment, the first enzyme comprises a lipase. In one embodiment, the second enzyme comprises a horseradish peroxidase. In one embodiment, the polymer comprises poly L-ascorbyl methyl methacrylate.

In one embodiment, the present invention contemplates a device, comprising: a) a plurality of microchannels arranged in a symmetric branching configuration, wherein said microchannels have an inlet and an outlet; b) a plurality of loading decks fluidly connected to said microchannel inlet; and c) a plurality of reaction chambers fluidity connected to said microchannel outlet. In one embodiment, the reaction chambers further comprise a microcomb filter. In one embodiment, the loading decks are selected from the group including, but not limited to, a substrate loading deck and a reactant loading deck. In one embodiment, the microchannel outlet further comprises at least one capillary check valve. In one embodiment, the symmetric microchannel branching configuration creates a plurality of mixing areas, wherein said mixing areas comprise a Y shape.

### DEFINITIONS

The term “flow manipulating component”, as used herein, refers to any component of a fluidic enzymatic cascade system capable of changing the fluid direction, altering the fluid velocity, and/or altering the fluid volume. For example, these components include, but are not limited to, microchannels, mixing areas, reaction chambers, microcomb filters and/or check valves.

The term “reaction”, as used herein, means any reaction involving inorganic, organic, or biochemical reactants. For example, at least one first chemical reactant may react with itself, or a second chemical reactant, to form a polymer (i.e, polymerization) that is catalyzed by a biochemical reactant (i.e., for example, an enzyme). In another embodiment, a polymer is broken down (at least partially) to create monomers.

The term “channels”, as used herein, are pathways through a medium (e.g., silicon) that allow for movement of liquids and gasses. Channels thus can connect other components, i.e., keep components “in liquid communication.” For example, “microfluidic channels” or “microchannels” are channels configured (in microns) so as to accommodate small solution volumes (i.e., nanoliters). While it is not intended that the present invention be limited by precise dimensions of the channels or precise solution volumes, illustrative ranges for channels and microdroplets are as follows: the channels can be between 0.35 and 50 μm in depth (preferably 20 μm) and between 50 and 1000 μm in width (preferably 500 μm), and the volume of the solutions can range between approximately one nanoliter (1 nl) and one milliliter (1 ml) but preferably between ten nanoliters (10 nl) and hundred microliters (100 μl).

The term, “microchannel inlet”, as used herein, refers to a terminal opening of a microchannel wherein a fluid enters the microchannel. For example, a microchannel inlet may be fluidly connnected to a loading deck wherein an introduced substrate and/or reactant passes through the loading deck and into the microchannel.

The term, “fluidic communication” or “fluidly connected” refers to any configuration of microchannels and/or microdevice components that allow for movement of liquids and gasses. Microchannels thus can connect other microdevice components thereby keeping “in communication” and more particularly, “in fluidic communication” and still more particularly, “in liquid communication”.

The term, “microchannel outlet”, as used herein, refers to a terminal opening of a microchannel wherein a fluid exits the microchannel. For example, a microchannel outlet may be fluidly connected to a reaction chamber wherein a moving reaction mixture passes through the microchannel outlet and into the reation chamber.

The term “pneumatic air pressure”, as used herein, refers to a force exerted upon a solution to create a flowing microfluidic stream within a microchannel.

The term “pumping system”, as used herein, refers to any component capable of applying air pressure into the fluidic enzymatic cascade system. For example, a pumping system includes, but is not limited to, a pneumatic air injection device.

The term, “hydrophilicity-enhancing compounds”, as used herein, are those compounds or preparations that enhance the hydrophilicity of a component, such as the hydrophilicity of a microchannel. An increase in hydrophilicity may include, but is not limited to, increasing the polar nature and/or presence of electrophilic groups on and/or within a microchannel. For example, Rain-X™ anti-fog is a commercially available reagent containing glycols and siloxanes in ethyl alcohol.

The term, “initiating a reaction”, as used herein, means causing a reaction to take place. Reactions can be initiated by any means (e.g., heat, wavelengths of light, addition of a catalyst, etc.) including, but not limited to, simply mixing of reactants.

The term “liquid barrier” or “moisture barrier”, as used herein, is any structure or treatment process on existing structures that prevents solution leakage and/or damage to a microfluidic device. In one embodiment of the present invention, the liquid barrier comprises a first silicon oxide layer, a silicon nitride layer, and a second silicon oxide layer.

The term “mixing”, as used herein, refers to the bringing together of at least two reactants. It is not intended that “mixing” be limited by degree. For example, In one embodiment, mixing creats a uniform distribution of components (e.g., reactants, products, or the like), while in other embodiments mixing may not create a uniform distribution (e.g., in the case where mixing initiates a reaction and reactants are used up). Such mixing may involve, but is not limited to, solutions, powders, crystals, etc.

The term “substrate”, as used herein, refers to any compound capable of mixing and reacting with other reactants to form a product. For example, an antioxidant may be a substrate wherein the antioxidant becomes covalently bound to a monomer during a polymerization process.

The term “reactant”, as used herein, refers to any compound capable of mixing and reacting with other compounds (i.e., for example, itself or another reactant and/or substrate). For example, such reacting may include, but is not limited to, the polymerization of a monomer to form a polymer. Any enzymatic cascade synthesis system may include a plurality of reactants to support a plurality of enzymatic reactions, wherein each enzymatic reaction occurs in separate reaction chambers in a sequential manner. Each reactant may, or may not, be introduced into the enzymatic cascade system through a separate loading deck.

The term “screening”, as used herein, refers to a separation of fluid elements by chemical or physical characteristics. For example, chemical characteristics may include, but are not limited to, ionic charge, hydrophobicity, and/or steric bulk. For example, physical characteristics may include, but are not limited to, monomeric elements, polymeric elements, molecular weight, and/or sedimentation rates.

The term “loading deck”, as used herein, refers to any reservoir within a fluidic enzymatic cascade system having an external opening for the introduction of a substrate and/or a reactant.

The term “reaction chamber”, as used herein, refers to any reservoir that is roughly rectangular which allows for interaction among chemicals. Optionally, a reaction chamber may be exposed to radiant heating from above or below, and optical/or observation and measurements. A reaction chamber may also comprise other enzymatic cascade system elements including, but not limited to, a separation element (i.e., for example, a microcomb filter). The reaction chamber may also connect to cross microchannels thereby allowing the diversion of a sample (or a portion thereof) from the main microchannel to a side microchannel.

The term “central microcomb filter”, as used herein, refers to a series of parallel and straight microchannels (i.e., for example, ˜20 microns wide) that can provide a physical filtering of sized particles as well as surface interactions (i.e., for example, when the microchannels are coated or filled with another filtering material). A central microcomb filter may also be in fluidic communication with a plurality of cross-channels for transport of a sample to reaction chambers for further chemical processing or detection. A central microcomb comprises a straight microchannels that have the advantage of minimizing clogging.

The term “side microcomb filter”, as used herein, refers to a series of parallel and bent microchannels. In some embodiment, a side microchannel may be configured either above or below a central microcomb filter, or both. The side microcomb filter is advantageous for maximizing surface area and smoother microflow characteristics the term “polymer”, as used herein, refers to any chemical compound or mixture of compounds formed by polymerization comprising repeating structural units (i.e., for example, monomers).

### BRIEF DESCRIPTION OF THE FIGURES

FIG. 1 presents one embodiment of a microfluidic enzymatic cascade microchip.

FIG. 2 presents one embodiment of a “Y” mixing channel.

FIG. 3 presents one embodiment of a PDMS checking valve (boxed area with outset)

FIG. 4 presents one embodiment of a plurality of microchip loading decks.

FIG. 5 presents one embodiment of a rapid separation microcomb filter.

FIG. 6 presents exemplary data showing representative flow fields in a microcomb separation filter.

FIG. 7 presents one embodiment of a pneumatic control device using radiant heating for non-contact thermal control.

FIG. 8 presents exemplary MALDI-TOF data of a synthesized 2000 mass (m)/charge (z) ratio L-ascorbyl-4-vinylbenzoate polymer. X Axis: m/z ratio. Y Axis: Relative Signal Intensity. Right Hand Box: Polymer signal. Left Hand Box: Precursor signal. Structure of identified polymer is shown.

FIG. 9 presents exemplary ¹H NMR data (300 MHz, DMSO-d₆) of a synthesized L-ascorbyl-4-vinylbenzoate polymer.

FIG. 10 presents one embodiment of an enzymatic synthetic and polymerization pathway of L-ascorbyl-4-vinylbenzoate. i) trifluoroethanol, 1,3-dicyclohexylcarbodimide, tetrahydrofuran, and 4-(dimethylamino)pyridine (25° C.); ii) ascorbic acid, C. antartica lipase (immobilized), and 1,4-dioxane (60° C.); iii) horseradish peroxidase, hydrogen peroxide, 2,4-pentanedione, methanol, and water.

FIG. 11 presents exemplary data showing the adhesion energy density (Y Axis: mJm⁻²) for PDMS (12.5:1) cantilever beams (* for RT; Δ for 60° C. preheat) as a function of step height (X Axis).

FIG. 12 presents exemplary data showing the adhesion energy density (Y Axis: mJm⁻²) for PDMS (10:1) cantilever beams (* for RT; Δ for 60° C. preheat) as a function of step height (X Axis).

FIG. 13 presents several embodiments of separation components. 1: No filter. 2: Low pass channel separation. 3: Medium pass channel separation. 4: High pass channel separation. 5: Medium pass well separation. 6: High pass channel filtration.

FIG. 14 presents one embodiment of a plurality of loading decks fabricated by PDMS casting.

### DETAILED DESCRIPTION OF THE INVENTION

The present invention is related to general enzymatic reactions, especially those involving the synthesis of new polymers or the (at least partial) breakdown of a polymer into monmers. This system and technology can be broadly used in various areas of polymer synthesis research, microfluidic engineering, elucidating metabolic relationships among pathways related to metabolic engineering and pharmaceuticals development. In one embodiment, a microfluidic device produces a polymer by enzymatic cascade.

The miniaturization of certain chemical and physical processes (i.e., for example, a single step enzymatic trans-esterification, or horseradish peroxidase reaction) is both possible and useful. In one embodiment, the present invention contemplates a method to produce polymers using a microfluidic device through a multi-step, enzymatic process. In one embodiment, the present invention contemplates a device capable of supporting a variety of enzymatic-reactions. In one embodiment, the device supports an enzymatic reaction to produce customized functional polymers.

In one embodiment, the present invention contemplates a microfluidic design for polymerization that integrates mixing, reactions, and multiple fluid sample manipulations on a single device. In one embodiment, the device is disposable. In another embodiment, the device is low-cost. In one embodiment, the device performs hundreds of experiment. In another embodiment, the device performs thousands of experiments. Although it is not necessary to understand the mechanism of an invention, it is believed that the primary value of this approach is that process optimization of variables such as time, temperature, and concentration can be performed in a much shorter time frame and at a lower cost.

### I. The Microfluidic Design System

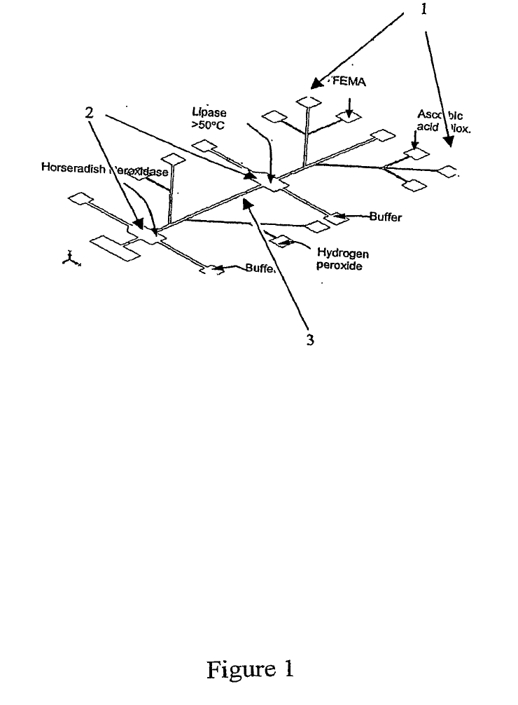

A microfabricated enzyme polymerization system comprises an on-chip, disposable, no moving parts (i.e., for example, monolithic), and pumping system which allows generation of a newly polymerized enzymatic products. The devices used here are small and monolithic in nature and thus reduce chemical use, and increased throughput of approximately 100-fold over corresponding macroprocesses. In one embodiment, the present invention contemplates a microfluidic design system comprising an enzymatic polymer synthetic cascade comprising a multi-step, cascading reaction. (See FIG. 1). In one embodiment, the system comprises plurality of loading decks (1) in fluidic communication with a plurality of reaction chambers (2) using a symmetric microchannel branch design (3). In one embodiment, a sample is injected into a loading deck by a syringe pump. In one embodiment, the sample comprises a volume of between approximately 0.01-100 microliters, preferably, 0.1-10 microliters, and more preferably 1-5 microliters. In one embodiment, the sample comprises a volume of approximately 2.5 microliters. In one embodiment, the system cascades the sample (i.e., for example, a mixture of reactants and/or intermediate products) from a first reaction chamber to a second reaction chamber to sequentially perform a biosynthetic process.

In some embodiments, a microfluidic design system comprises a microchip including, but not limited to: i) the absence of moving internal parts, with all of the microfluidic components integrated on a single, monolithic chip to improve reliability over conventional systems; ii) a “Y” shaped mixing channel rather than the more commonly used Mixing “T”; iii) one or more microcomb filtering arrays, iv) one or more unidirectional, passive check valves, capable of creating at least a 5 kPa pressure barrier involving the capillary forces of the cascading fluid; v) MEMS based silicon micromachining; vl) polydimethylsiloxane (PDMS) polymer microcasting, vii) oxygen plasma bonding capable of creating a low cost microsystem.

In operation, one embodiment of the chip handles polymerization by using mildly selective enzymatic methods. In one embodiment, a first enzymatic reaction covalently couples a primary hydroxyl group of a compound (i.e., for example, ascorbic acid) with a monomer (i.e., for example, a vinyl or methyl methacrylate monomer). In one embodiment, a second enzymatic reaction polymerizes the monomer (i.e., for example, horseradish peroxidase polymerizes a methyl methacrylate monomer yielding an ascorbic acid polymer). Although it is not necessary to understand the mechanism of an invention, it is believed that this low-cost device reduces the time required by a conventional process by 95% (i.e., for example, producing a polymer within minutes), while using only 1% of the material needed in a traditional sample. A final polymerized antioxidant biomaterial product, as exemplified above, is useful in polymethyl methacrylate (PMMA) or polystyrene polymers because the ascorbic acid will improve the effectiveness of therapeutic and pharmaceutical compositions, personal care products, and increase food quality.

In one embodiment, the enzymatic polymer synthesis system further comprises a mixer. In one embodiment, the mixer is capable of combining a polymer with a monomer. The system further comprises a sequential transport process capable of moving intermediate products from a first reactor to a second reactor. In one embodiment, the transport process comprises at least one flow manipulating component including, but not limited to, channels and valves. In one embodiment, the valve is a directional check valve. In one embodiment, the channel comprises a separation component capable of removing a functionalized polymer from the system and separating out unreacted compounds.

This system provides advantages that provide a smaller, faster version for various processes related to biochemical experimentation including, but not limited to, mixing, reaction, and separation. Although it is not necessary to understand the mechanism of an invention, it is believed that synthesis times are on the order of seconds and/or minutes because flowing microfluidic have nanoliter volumes and the devices used are small and monolithic in nature.

### II. The Microchip

The present invention introduces a ‘Microenzymatic Polymer Synthesis Microchip’ that may simplify the job of the analytical chemist, and to deliver a significant breakthrough in the art. Nano-liter chip devices, as contemplated herein, can dramatically reduce chemical consumption, and processing time, as well as allow for precise sample manipulation. This concept proposes the full incorporation of analytical procedures into flowing microfluidic systems. Carrier streams of fluids, rather than human hands, take over the role of sample transport between different sample manipulation steps. Flow paths may be defined by interconnected pieces of tubing and channels, arranged in such a way that the desired synthesis can be performed.

Miniaturization itself is an important research direction in the MEMS field. Burns et al., “An integrated nanoliter DNA analysis device” Science, 282:484-487 (1998); Manz et al. “Planar chips technology for miniaturization and integration of separation techniques into monitoring systems-Capillary electrophoresis on a chip” J. Chromatogr., 593:253-258 (1992); Jacobson et al., “High-speed separations on a microchip” Anal. Chem., 66:895-897 (1993); and Harrison et al., “Micromachining a miniaturized capillary electrophoresis-based chemical analysis system on a chip” Science, 261:895-897 (1993). In one embodiment, the present invention contemplates a microfluidic chip capable of performing operations associated with an enzymatic polymer synthesis process. This technology is believed to have many advantages including, but not limited to, manufacturing simplicity, affordability, scalability, flexibility, energy efficiency and environmental sustainability. In one embodiment, the chip further comprises procedures to purify the synthesized polymers. In one embodiment, the purification procedures include, but are not limited to, selective coatings, selective filters, functional material assessments using additional devices placed in series and/or parallel with the microfluidic synthesis cascade.

In one embodiment, a microchip comprises five (5) components capable of performing five (5) separate process requirements, including, but not limited to: i) a first mixing area; ii) a screening area; iii) a capillary valve fluid manipulating area; iv) a second mixing area; and v) a separation area. Although it is not necessary to understand the mechanism of an invention, it is believed that these five (5) components may be arranged in five (5) consecutive areas thereby allowing the process to cascade from area to area.

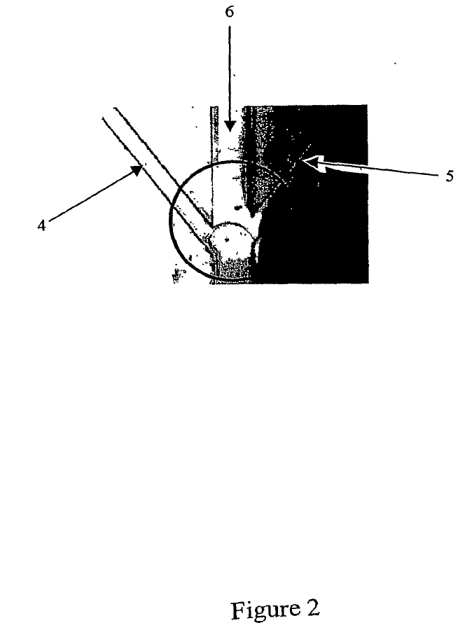

Many microfluidic microchip designs and potential chemical input locations are possible. For the purposes of illustration only the following device is exemplified. In one embodiment, the first and/or second mixing areas comprise a mixing “Y” thereby providing improved efficiency and reducing mixing lengths. (See FIG. 2) In one embodiment, the mixing areas comprise an intersection of a first side microchannel (4) and a second side microchannel (5) with a main channel (6) thereby producing a lamellar fluid configuration and a more rapid mixing.

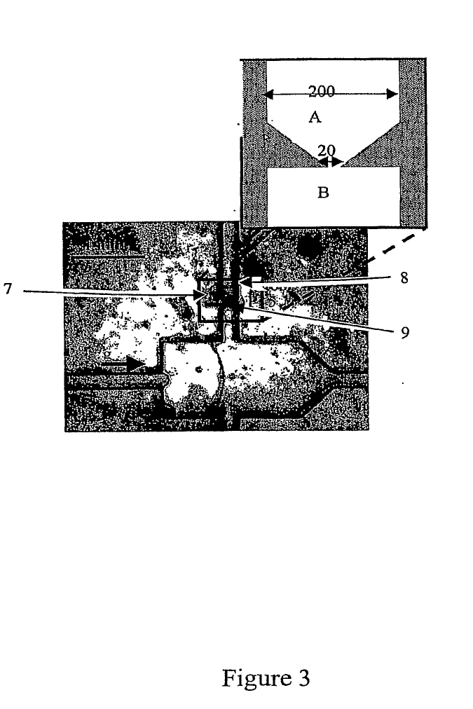

In one embodiment, the microfluidic microchip comprises a monolithic chip (i.e., for example, has no moving mechanical parts). In one embodiment, a unidirectional capillary check valve operates as a result of capillary force and/or pressure (i.e., for example, liquid-gas interface capillary effects). (See FIG. 3). In one embodiment, a capillary check valve (7) comprises a microchannel having a first diameter (8) and a second diameter (9), wherein the second diameter is between approximately 10-1,000 percent smaller than the first diameter. In one embodiment, the second diameter is at least fifty (50) percent smaller than the first diameter. In one embodiment, the second diameter is at least one hundred (100) percent smaller than the first diameter. In one embodiment, the second diameter is at least two hundred (200) percent smaller than the first diameter.

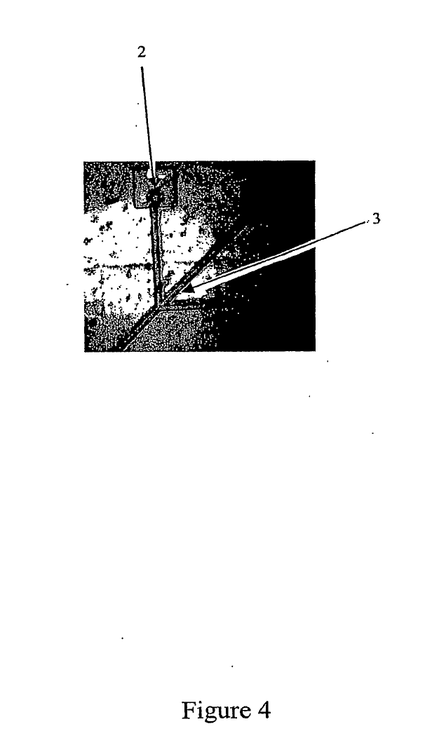

In one embodiment, the microfluidic microchip comprises a sample loading sequence that is controllable by a plurality of loading decks (1) in fluidic communication with a symmetric branching of microchannels (3). (See FIG. 4) In one embodiment, a loading deck comprises a reservoir having an external opening and an internal opening, wherein the internal opening is in fluidic communication with a fluid microchannel.

In one embodiment, the microfluidic microchip comprises a symmetric branch design capable of controlling flow stability. (See FIG. 1).

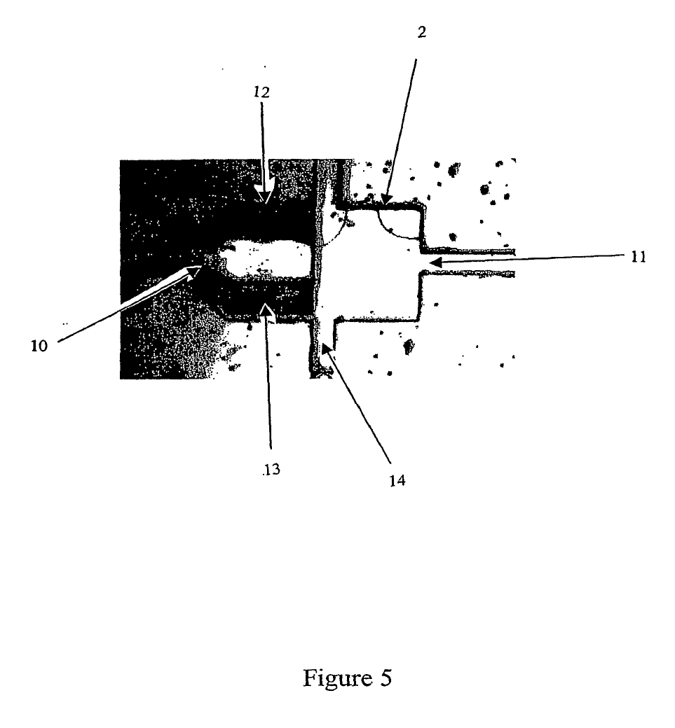

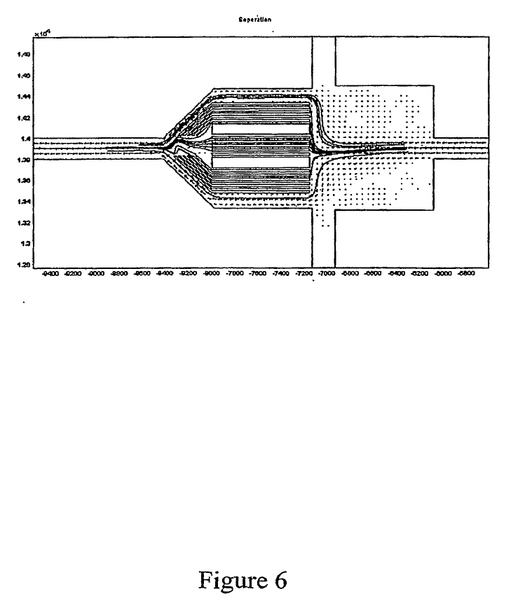

In one embodiment, the microfluidic microchip comprises a reaction chamber, In one embodiment, the reaction chamber (2) comprises an inlet microchannel (10), a microchannel (11), and at least one cross microchannel (14). In one embodiment, the chamber further comprises a side microcomb filter (12) comprising a plurality of filtration microchannels (13) in fluidic communication with a microchannel outlet (10). (See FIG. 5). In another embodiment, the filter comprises a central microcomb and at least one side microcomb. In one embodiment, the component comprises a central microcomb and two side microcombs. In one embodiment, the microcomb filtering component purifies the sample (i.e., for example, a synthesized polymer) using a plurality of microchannels by providing differential flow paths. (See FIG. 6). In one embodiment, the microcomb comprises a plurality of microchannels, wherein the microchannels may be of the same, or different diameters (i.e., for example, 20 μm. In one embodiment, the microchannels are arranged in an array. In one embodiment, the array comprises straight microchannels and bent microchannels. In one embodiment, the array comprises straight microchannels. In another embodiment, the array comprises bent microchannels. In one embodiment, the microcomb filtering component is in fluidic communication with a reaction chamber. In one embodiment, the microcomb filtering component further comprises at least one side channel, wherein the side channel is in fluidic communication with at least one microchannel. In one embodiment, the microcomb filtering array provides a screening process.

Although it is not necessary to understand the mechanism of an invention, it is believed that the principals of microfluidic microcomb filtering combines both physical separation and chemical filtering. For example, if the products of the reactions in the chamber are larger than the reaction components, then a cross-flow in the chamber may produce a physical separation with the smaller chemical components flowing out of the chamber, leaving more of the product in the chamber. Verification of these principles was achieved by conducting a microfluidic analysis to ensure that the flow was consistently in the desired direction (i.e., unidirectionally outward from the reaction chamber/microcomb) and that there is no back-flow into any of the microcombs.

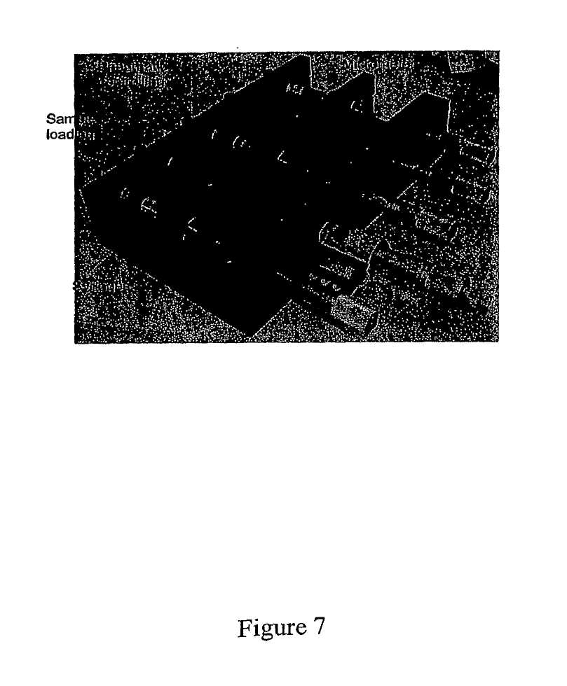

In one embodiment, the microfluidic microchip is in fluidic communication with pneumatic air compression device. See FIG. 7. In one embodiment, the microchip comprises a sample that is moved by pnematically compressed air.

Although it is not necessary to understand the mechanism of an invention, it is believed that a microfluidic microchip reduces the time required by a conventional process to produce a synthesized polymer by 95%, while using only 1% of the material. For example, many such polymerization reactions using a microscale version of enzymatic polymer synthesis are contemplated herein. (infra, and for example, see Example I).

The microenzymatic polymer synthesis chip as contemplated herein is an innovation in microenzymatic research. Although it is not necessary to understand the mechanism of an invention, it is believed that the physics and processes employed for macroscale systems do not work well for microscale systems. From this perspective, microenzymatic polymer synthesis is in some respects, a new research area, different from macroscale enzymatic synthesis, as the size of the processing channels is reduced to microns and microfluidic considerations need to be addressed.

In one embodiment, the microfluidic enzymatic synthesis devices are small and monolithic in nature. These characteristics can be achieved in a disposable 3-D system by utilizing oxygen plasma surface treatment to bond Pyrex® to polydimethylsiloxane microfluidic channels with a cross-section on the order of microns. Qin et al., “Microfabrication, Microstructures and Microsystems”, Microsystem Technology in Chemistry and Life Sciences, 194:1-20 (1998); Roberts et al., “Using Mixed Self-Assembled Monolayers Presenting RGD and (EG)₃OH Groups To Characterize Long-Term Attachment of Bovine Capillary Endothelial Cells to Surfaces”, J. Am. Chem. Soc. 120:6548-6555 (1998); Terfort et al., “Self-Assembly of an Operating Electrical Circuit Based on Shape Complementarity and the Hydrophobic Effect”, Adv. Materials 10:470-473 (1998); and Zhao et al., “Fabrication of microstructures using shrinkable polystyrene films”, Sensors and Actuators, A: Physical 65:209-217 (1998).

In one embodiment, the microfluidic enzymatic cascade system is capable of supporting multi-enzymatic synthesis steps to generate new polymers. This system could also be used for other enzyme reaction applications, having advantages including, but not limited to, small volume mixing and chemical sample detection. One system that is contemplated herein may be used for binding assays (i.e., for example, enzymes, proteins, or phage binding), nucleic acid amplification, and/or nucleic acid sequencing.

Although it is not necessary to understand the mechanism of an invention, it is believed that one advantage of miniaturization is the dramatically increased performance of the system, regardless of the size of surrounding instrumentation. In addition, it is believed that since a microchemical system is smaller, it is inherently safer and capable of shorter thermal and chemical response times. Therefore, further miniaturization of chemical systems solve some limitations exhibited by conventional systems (such as batch vessels), and can improve, enable and potentially revolutionize chemical systems. One example of this capability comprises an enzymatic cascade chip capable of polymerizing an ascorbic acid polymer with an enzymatic catalysis reaction at the microscale.

Another advantage of the presently contemplated microfluidic microchip dramatically reduces chemical consumption. PDMS is used to achieve high volume production, low cost, and contamination free capability. PDMS Microsystems are also disposable.

### III. Fabrication

The use of planar fluidic devices for performing small-volume chemistry was first proposed by analytical chemists, and established “miniaturized total chemical analysis system” (μTAS). Manz et al., “Miniaturized total chemical analysis system: a novel concept for chemical sensing,” Sens. Actuators, B1:244-248 (1990). More recently, the μTAS field has begun to encompass other areas of chemistry and biology. To reflect this expanded scope, the broader terms “microfluidics” and “lab-on-a-chip” are now often used in addition to μTAS. The first successful demonstration of chip-based analysis involved the fast separation of fluorescent dyes and fluorescent labeled amino acids by capillary electrophoresis. Man et al., “Microfluidic plastic capillaries on silicon substrates: A new inexpensive technology for bioanalysis chips,” IEEE MEMS Conference Nagoya, Japan, pg. 311-316, (February 1997).

Currently most microfluidic technology relies on micromachining (at least to some extent) to produce microflow systems based on interconnected micrometer-dimensioned channels. Advances in microelectromechanical systems (MEMS), however, has improved the art of micromachining. For example, it is possible to impart higher levels of functionality by making features in different materials and at different levels within a microfluidic device. The integration of more functions into chips for purposes as diverse as heating, fluidic controlling, electrochemical detection, and pumping is becoming more common. Losey et al., “Design and fabrication of fluidic devices for multiphase mixing and reaction” J. Microelectromech. Syst., 11:709-717 (2002); and Zou et al., “Thermal Effects in Plasma Treatment of Patterned PDMS for Bonding Stacked Channels” Materials Research Society 782:A5.5.1 (2004).

A. Microfluidic Devices

The length scales of microdevices are short thereby resulting in a small Reynolds number and a laminar flow. One advantage of the microdevice's short length is that when two or more streams are contacted in a homogenous system the flow is relatively stable. Although it is not necessary to understand the mechanism of an invention, it is believed that these short length scales and resulting lamellar stream enables rapid diffusion mixing for many applications including, but not limited to, kinetic studies or reaction-rate limited operation of fast reactions. A further advantage of the MEMS processes is that new materials and new approaches for the fabrication of microfluidic devices are possible.

Consequently, the developing technology of microfluidics has improved the interaction between the MEMS and biochemical analysis. For example, polydimethylsiloxane (PDMS: Dow Corning, sylgard with repeat unit [—Si(CH₃)₂O—]) coatings and PDMS thermal treatments may be used to integrate large-scale analysis chips and make a microfluidics system simple, reliable, and disposable. Zou et al., “Thermal Effects in Plasma Treatment of Patterned PDMS for Bonding Stacked Channels” Materials Research Society 782:A5.5.1 (2004). Since the majority of presently available devices do not feature elements such as, an on-board pump, valves or flow sensors, a very precise flow control is required in all branches of the fluid networks. In one embodiment, the present invention contemplates a symmetric branch design and an on-channel valve design in the enzymatic cascade chip capable of on-chip flow control.

B. Channel Construction

Silicon has compatible fabrication characteristics for the construction of microfluidic channels on a microchip. The principal modern method for fabricating semiconductor integrated circuits is the so-called planar process. The planar process relies on the unique characteristics of silicon and comprises a complex sequence of manufacturing steps involving deposition, oxidation, photolithography, diffusion and/or ion implantation, and metallization, to fabricate a “layered” integrated silicon circuit device. See e.g., W. Miller, U.S. Pat. No. 5,091,328 (herein incorporated by reference).

1. Photolithography

For example, oxidation of crystalline silicon results in the formation of a surface layer of silicon dioxide. Photolithography can then be used to selectively pattern and etch the silicon dioxide layer to expose a portion of the underlying material. Of course, the particular fabrication process and sequence used will depend on the desired characteristics of the device. Today, one can choose from among a wide variety of microdevice designs whether or not the device comprises an integrated circuit.

For example, microchannels may be prepared on a 500 μm thick glass wafer (i.e., for example, a microchip) (Dow Corning 7740) using standard aqueous-based etch procedures. The initial glass surface can be cleaned to receive two layers of electron beam evaporated metal (20 nm chromium followed by 50 nm gold). Photoresist Microposit 1813 (Shipley Co.) is then applied at 4000 rpm for 30 seconds and patterned using a first mask and subsequently developed. The metal layers may then be etched in a chromium etchant (i.e., for example, ¹⁴Cr, Cyantek Inc.) and a gold etchant (i.e., for example, Gold Etchant TFA, Transene Co.) until the pattern is clearly visible on the glass surface. The accessible glass can then be etched in a solution of hydrofluoric acid and water (i.e., for example, 1:1; v/v). Etch rates are estimated using test wafers, with the final etch typically giving channel depths of 20 to 30 μm. For each wafer, the depth of the finished channel can be determined using a surface profilometer. A final stripping (PRS-2000, J. T. Baker) removes both the remaining photoresist material and the overlying metal.

As described in additional detail below, some embodiments of the present invention contemplate channels etched on glass and are bonded in a multi-part construction approach (i.e., for example, stacked channels) using an adhesive (i.e., for example, an optical adhesive SK-9 Lens Bond, Sumers Laboratories, Fort Washington, Pa.). Optical adhesive bonding is cured under an ultraviolet light source (365 nm) for 12 to 24 hours.

Loading deck elements may be fabricated as follows. A silicon wafer (p-type, 18-22½-cm, {100}, boron concentration Å 10¹⁵ cm⁻³) can be used to grow SiO₂ thermal oxide (1 μm). Photoresist (AZ-5214-E, Hoescht-Celanese) is applied and spun at 3000 rpm, 30 seconds. The resist is patterned using a mask and developed. Reactive ion etch (RIE, PlasmaTherm, Inc.) can be performed to a preferred depth (i.e., for example, 0.35 μm) into the SiO₂ layer at the following conditions: CHF₃, 15 sccm (standard cubic centimeters per minute); CF₄, 15 sccm; 4 mTorr, DC bias voltage of 200 V, 100 W, 20 minutes. The etch depth can then be measured by profilometer. The resist is then lifted off by development using Microposit 1112A remover in solution (Shipley Co.). RIE can also be used to etch contact holes using a second mask (CHF₃, sccm; CF₄, 15 sccm; 4 mTorr; and DC bias voltage of 200 V, 100 W, 120 minutes).

As shown in FIG. 1, In one embodiment, the loading deck elements are arrayed as groups (i.e., for example, 500 μm wide) merging into one channel, thereby forming a “Y” intersection. These channels may be uniformly etched (i.e., for example, 500 μm wide and approximately 20 μm deep).

Prior to performing enzymatic polymerization within a microfluid stream, the channels are preferably treated by washing with base, acid, buffer, water and a hydrophilicity-enhancing compound, followed by a relatively high concentration solution of non-specific protein. In a preferred embodiment, the channels are washed with approximately 100 μl each of the following solutions in series: 0.1N NaOH; 0.1N HCl; 10 mM Tris-HCl (pH 8.0), deionized H₂O, Rain-X Anti-Fog (a hydrophilicity-enhancing compound commercially available from Unelko Corp., Scottsdale, Ariz.), and 500 μg/μl bovine serum albumin (non-specific protein commercially available in restriction enzyme grade from GIBCO-BRL). Alternatively, a channel may be made hydrophobic, either entirely or in part, by coating with Teflon®.

2. Poly(dimethylsiloxane) Microcasting

Fabrication of devices using polymers reduces the time, complexity, and cost of prototyping and manufacturing. Soper et al., “Polymeric Microelectromechanical Systems” Anal. Chem. 72:642A-651A (2000); and Becker et al., “Polymer Microfabrcation Methods for Microfluidic Analytical Applications” Electrophoresis 21:12-26 (2000). For example, poly(dimethylsiloxane) (PDMS) is a polymer compatible with the fabrication of microfluidic devices. McDonald et al., “Fabrication of Microfluidic Systems in Poly(dimethylsiloxane). Electrophoresis 21:27-40 (2000). Fabrication of channel systems comprising PDMS is particularly straightforward since it can be cast against a suitable mold having a resolution of less than 0.1 μm. PDMS is also more than a structural material: its chemical and physical properties make possible fabrication of devices with useful functionality. (See Table 4). McDonald et al., “Poly(dimethylsiloxane) as a material for fabricating microfluidic devices” Accounts Of Chemical Research 35:491-499 (2002).

TABLE 4
Physical and Chemical Properties of PDMS

| Property | Characteristic | Consequence |
|---|---|---|
| Optical | transparent; UV cutoff 240 nm | optical detection from 240 to 1100 nm |
| Electrical | insulating; breakdown voltage | allows embedded circuits; intentional |
| | @ 2 × 10⁷ V/m | breakdown to open connections |
| Mechanical | elastomeric; tunable Young's | conforms to surfaces; allows actuation by |
| | modulus typical value of ~750 | reversible deformation; facilitates release from |
| | kPa | molds |
| Thermal | insulating; thermal | can be used to insulate heated solutions does not |
| | conductivity 0.2 W/(m · K); | allow dissipation of resistive heating from |
| | coefficient of thermal | electrophoretic separation |
| | expansion 310 μm/(m · ° C.) | |
| Interfacial | low surface free energy ~20 | replicas release easily from molds; can be |
| | erg/cm² | reversibly sealed to materials |
| Permeability | impermeable to liquid water; | contains aqueous solutions in channels; allows |
| | permeable to gases and | gas transport through the bulk material; |
| | nonpolar organic solvents | incompatible with many organic solvents |
| Reactivity | inert; can be oxidized by | unreactive toward most reagents; surface can be |
| | exposure to a plasma; Bu₄N⁺F⁻ | etched; can be modified to be hydrophilic and |
| | ((TBA)F) | also reactive toward silanes; etching with |
| | | (TBA)F can alter topography of surfaces |
| Toxicity | nontoxic | can be implanted in vivo; supports mammalian |
| | | cell growth |

PDMS microcasting begins with soft lithography that creates a replica mold (i.e., a master copy) and is useful for rapid prototyping. The PDMS may be applied as two components, a base and a curing agent. Silicon hydride groups present in the curing agent react with vinyl groups present in the base and form a cross-linked, elastomeric solid. To produce a replica mold, the two components are mixed together (i.e., for example, 10:1 (v/v) base:curing agent), then the liquid pre-polymer is poured over a master copy, and the microcasting is left to cure. While curing, the liquid PDMS pre-polymer conforms to the shape of the master and replicates the features of the master with high fidelity (i.e., for example, on the order of approximately 10 nm). After the microcast is cured, the low surface free energy and elasticity of PDMS allows it to release from master copy without damaging the master or microcast molding.

Master copies can be obtained by a range of previously reported methods. Becker et al., “Polymer Microfabrcation Methods for Microfluidic Analytical Applications” Electrophoresis 21:12-26 (2000); Duffy et al., “Rapid Prototyping of Microfluidic Systems in Poly(dimethylsiloxane)’ Anal. Chem. 70:497-44984 (1998); Anderson et al., “Tabrication of Topologically Complex Three-Dimensional Microfluidic Systems in PDMS by Rapid Prototyping” Anal. Chem. 72:3158-3164 (2000); DeBusschere et al., “Portable Cell-Based Biosensor System Using Integrated CMOS Cell-Cartridges” Biosens. Bioelectron. 16:543-556 (2001); McDonald et al., “Prototyping of Microfluidic Devices Using Solid-Object Printing” Anal. Chem. 74:1537-1545 (2002); Love et al., “Microscope Projection Photolithography for Rapid Prototyping of Masters with Micron-Scale Features for Use in Soft Lithography” Langmuir 17:6005-6012 (2001); Effenhauser et al., “Integrated Capillary Electrophoresis on Flexible Silicone Microdevices: Analysis of DNA Restriction Fragments and Detection of Single DNA Molecules on Microchips” Anal. Chem. 69:3451-3457 (1997); McKnight et al., “Electroosmotically Induced Hydraulic Pumping with Integrated Electrodes on Microfluidic Devices” Anal. Chem. 2001, 73:4045-4049 (2001); Bernard et al., “Micromosaic Immunoassays” Anal. Chem. 73:8-12 (2001). Commonly, the method starts with using a high-resolution transparency as a photomask for generation of the master by photolithography (FIG. 1). A transparency resolution greater than 20 μm is adequate for most microfluidic applications.

One advantage of PDMS is that it can seal to itself or to other surfaces, reversibly or irreversibly, and without distortion of the channels. The self-sealing nature of PDMS channels makes it a preferable material when compared to other channel materials that require active scaling including, but not limited to, glass, silicon, quartz, or thermoplastics. Further, PDMS that has been molded against a smooth surface can conformally contact other smooth surfaces, even if they are nonplanar because PDMS is elastomeric.

A reversible seal provided by simple van der Waals contact is watertight but cannot withstand pressures greater than ˜5 psi. Adhesive tapes, silicone, Parafilm®, or cellophanes may also reversibly seal a PDMS channel. Cellophane tape, however, provides only a temporary seal but silicone adhesive tape makes a much stronger seal, is waterproof, and provides “a fourth PDMS wall”.

To form an irreversible seal, PDMS, and perhaps a second surface, may be exposed to an air plasma for approximately 1 min. Although it is not necessary to understand the mechanism of an invention, it is believed that this treatment generates silanol groups (Si—OH) on the surface of the PDMS by the oxidation of methyl groups. Surface-oxidized PDMS can seal to many compounds including, but not limited to, PDMS, glass, silicon, polystyrene, polyethylene, or silicon nitride, provided that these surfaces have also been exposed to an air plasma.

This sealing process involved bringing two surfaces into contact quickly (<1 min) after oxidation because the surface of the oxidized PDMS reconstructs in air. Contact with water or polar organic solvents maintains the hydrophilic nature of the surface indefinitely. It is further believed that oxidative sealing works best when the samples and chamber are clean, the samples are dry, the surfaces are smooth on the micron scale, and the oxidized surfaces are not mechanically stressed. Heating a weak seal in an oven at 70° C. can sometimes improve the strength of the seal.

An alternative method for irreversibly sealing involves adding an excess of the base to one slab of PDMS and an excess of curing agent to the other slab. Unger et al., “Monolithic Microfabricated Valves and Pumps by Multilayer Soft Lithography” Science 288:113-116 (2000). When these layers are brought into conformal contact and again cured, the seal that is formed is indistinguishable in physical properties from bulk PDMS. The method comprises careful alignment of two slabs, heat sealing, and can generate hydrophobic channels. Treatment with hydrochloric acid makes the channels slightly hydrophilic.

Introducing and recovering fluids (e.g., samples, reagents, or buffers) from PDMS microcasts can be accomplished by using compression fitting of polyethylene tubing. Holes slightly smaller than the outer diameter of the tubing are bored in the PDMS. When tubing is inserted a pressure is exerted on the PDMS and provides a waterproof seal. This method provides a reversible seal, since the tubing can be removed and replaced with minimal effort. The polyethylene tubing also conforms to syringe needles. This ability allows for syringes (and syringe pumps) to be coupled easily to microfluidic channels. It is also straightforward to make fluidic connections by using loading decks (i.e., for example, fluid reservoirs) that are accessible by pipet.

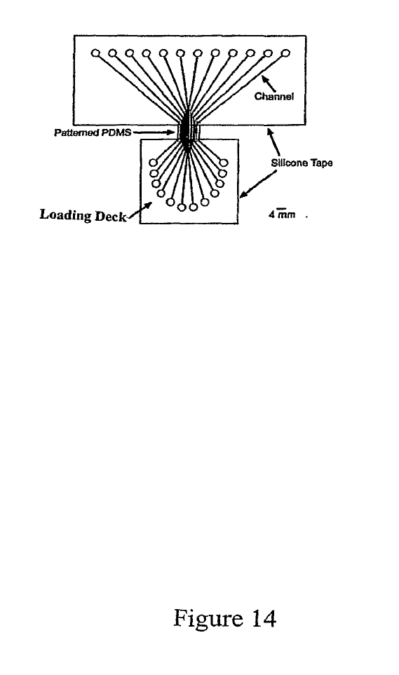

FIG. 14 shows a system designed for sample addition that uses twelve loading decks. These loading decks were molded to match the spacing of a standard 12-channel pipettor. Each of these reservoirs are connected to a microfluidic channel. In this particular example, since the channels are all the same length the flow rates are also the same in each channel. However, any combination of channel lengths may be constructed depending upon reaction assay requirements.

3. Solid Object Printing

A solid-object printer may used to produce masters for the fabrication of microfluidic devices in poly(dimethylsiloxane) (PDMS). These printers provide an alternative to photolithography for applications where features of >250 microns are needed. Solid-object printing is capable of delivering objects that have dimensions as large as 250×190×200 mm with feature sizes that can range from 10 cm to 250 microns. A 3-D design of a microfluidic device is created in a Computer Assisted Drawing (CAD) program. Subsequently, the CAD file is used by the printer to fabricate a master directly without the need for a mask. These printers can produce complex structures, including multilevel features (i.e. for example, stacked channels). Once a master is obtained, a PDMS replica may be fabricated by molding thereby allowing the fabrication a microfluidic device. The capabilities of this method include, but are not limited to, devices that contain multilevel and tall features, devices that cover a large area (approximately 150 cm²), and devices that contain nonintersecting, crossing channels. McDonald et al., “Prototyping of microfluidic devices in poly(dimethylsiloxane) using solid-object printing” Anal Chem 74:1537-1545 (2002).

C. Stacked Channels

Channel stacking can be considered to be a result of a “membrane sandwich”. This method allows the fabrication of complex systems of channels by stacking multiple, thin (i.e., for example, ˜100 μm) 2D layers. Since PDMS is transparent, visual alignment of the layers required to form 3D systems is usually straightforward (i.e., for example, by using a stereomicroscope). A “membrane sandwich” method may allow up to three levels of features to be present in a single layer. Membranes may be molded between two masters (i.e., a “top” master and a “bottom” master) by placing a small amount of liquid PDMS between the two masters and aligning them relative to one another. When pressure is applied, features on each master that contact one another form vias. The alignment of two oxidized layers of PDMS containing embedded channels forms multilevel channel systems.

One method to align multilayer systems uses solvent-assisted sealing. Kim et al., “Microfabricated PDMS Multichannel Emitter for Electrospray Ionization Mass Spectrometry” J. Am. Soc. Mass. Spectrom. 12:463-469 (2001). After removing the PDMS from a plasma cleaner, the oxidized surface may be covered with a film of polar solvents including, but not limited to, methanol, ethanol, or trifluoroethanols. Although it is not necessary to understand the mechanism of an invention, it is believed that these solvents act in at least three ways: i) they prevent instantaneous sealing when two layers are brought into contact; ii) they provide lubrication and allow the layers to be moved laterally relative to one another; and iii) they prevent the oxidized surface from reconstructing to a lower free-energy form before sealing is accomplished.

For a complete sealing, a device is heated thereby allowing the solvent to evaporate from between the layers of PDMS (i.e., for example, using a hot plate or an oven). Sealing with this method produces a bond equivalent to sealing immediately upon removal from the plasma cleaner and forms hydrophobic channels.

In one embodiment, the present invention contemplates preheating to obtain plasma oxidation (ashing) of patterned polydimethylsiloxane (PDMS) for Bio-MEMS applications. PDMS creates an irreversible seal to itself as well as strong seals with glass, silicon, and silicon nitride. This process activates the surface by producing hydroxyl groups that last for several minutes to allow bonding. Several channels can be stacked to create 3D systems for microfluidic applications using PDMS alone or in combination with other materials to develop hybrid systems. For PDMS, bonding temperatures typically occur at room temperature. Good bonding of PDMS to slides with a work adhesion on the order of 100 mJm⁻² may be obtained. Preheating the samples at 65° C. results in a significant increase in adhesive properties depending on the mixture composition. Processing temperature and chemical components effect both bond quality and adhesive properties.

Treatment of PDMS in plasma oxidation for approximately 1 minute results in the formation of hydroxyl groups that are changed from being hydrophobic to hydrophilic through a dehydration reaction. When the internal energy falls below a specific limit, the hydrophilic state will regroup to re-form the hydrophobic state.

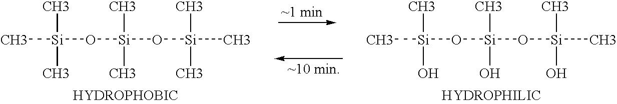

The hydrophilic PDMS can form chemical bonds with itself or other materials. Preheating the PDMS prior to ashing has several effects that includes, but is not limited to, reinforcing curing, softening the bulk material, and surface drying. The material may also preheated to the same temperature to limit the thermal stresses formed during subsequent cooling. Although it is not necessary to understand the mechanism of an invention, it is believed that plasma oxidation effects are improved since the surface reactions are temperature dependent.

Typically, in hard crystalline solids, bond strength energy is very high (i.e., for example, 500-2000 mJm⁻²), and in soft solids, such as polymers, it is very low (i.e., for example, 5-100 mJm⁻²). Measuring the adhesion force directly is difficult. Measurements of loads comprising pushing, pulling, shearing, or peeling have been used with various results. Surface energy measurements, however, usually yields consistent values. In this technique a blade of known thickness is inserted between the hard bonded elements to promote the formation of a crack, whose length is subsequently measured. In diffusion bonding, voids are the most common defects, which can be evaluated by a pressure burst test. For soft materials, however, a surface energy approach may be used. For example, a simple peeling technique in which a cantilever beam suspended over a material has as much of the length of the beam forced down to the material. Although it is not necessary to understand the mechanism of an invention, it is believed that the elastic forces of the cantilever pulls up the beam until an equilibrium point between elastic and adhesive forces is reached. The elastic bending energy and adhesion work being stored in this system is minimized, and the adhesion energy balances the elastic bending energy. See Example III.

D. Design and Fabrication of Reaction Chamber/Microcomb Filters

In one embodiment, the reaction chamber/filter components may be designed using L-EDIT® software. This type of computer program can layout a computer file from which a photolithography mask can be printed (i.e., for example, a transparency film). The mask can be used with Deep Reactive Ion Etching (DRIE) to create a silicon master mold with the design transferred to the wafer. PDMS microcasting can be used to create devices with the appropriate features of the reaction chamber/filter component. Although it is not necessary to understand the mechanism of an invention, it is believed that the etch depth of the DRIE step should be greater than 20 microns. In one embodiment, the DRIE etch depth is approximately 90 microns. It is further believed that microchannel widths should be greater than 20 microns.

### IV. Enzymatic Polymerization

Enzymatic polymerizations can produce product polymers obtained under mild reaction conditions without using toxic reagents. Therefore, enzymatic polymerization can be regarded as an environmentally friendly synthetic process of polymeric materials, providing a good example for achieving “green polymer chemistry”. Kaplan D., In: Biopolymers from Renewable Resources, Springer-Verlag Berlin Heidelberg New York, pp 249, 323 1998 (herein incorporated by reference). The target macromolecules for the enzymatic polymerization include, but are not limited to, polysaccharides, polyesters, polycarbonates, poly(amino acid)s, polyaromatics, and/or vinyl polymers.

In one embodiment, the present invention contemplates providing miniaturized devices for commercially feasible enzymatic polymer synthesis. For example, a low cost but highly efficient method to produce vitamin C enriched polymers has both scientific and market value. Vitamin C enriched polymers can alleviate harmful exposure to humans resulting from the excessive use and exposure to chemical antioxidants such as butylated hydroxy anisole (BRA) and butylated hydroxy toluene (BHT). The carcinogenic effects of BRA and BHT has prompted the Food and Drug Administration to limit their concentrations in food to 0.02%. However, antioxidants are also considered important in reducing aging-related phenomena by providing protection against free radicals. Thus, vitamin C and other natural antioxidants are considered as suitable substitutes for BHA and BHT. Nutraceutical supplementation with ascorbic acid may have an overall positive impact on public health because humans lack the ability to synthesize vitamin C due to loss of function in the gene coding for L-gulono-y-lactone oxidase. Therefore, vitamin C must be obtained from the diet.

When vitamin C (i.e., ascorbic acid) is utilized in commercial products it is prone to moisture-induced degradation resulting in brown discoloration. For example, 6-O-palmitoyl-ascorbic-acid is often chosen to provide antioxidant activity in fats and oils (since ascorbic acid is not fat soluble). One chemical process to prepare this compound involves an acid-catalyzed esterification of ascorbic acid resulting in the formation of mixtures of products with a preponderance of O-6 substitutions. However, undesirable by-products are produced due to the instability of ascorbic acid thereby leading to commercially unacceptable yields.

To overcome these problems, a strategy was developed to use mild and highly selective enzymatic methods to covalently couple the primary hydroxyl group of ascorbic acid with a vinyl monomer, followed by a second enzymatic reaction catalyzed by horseradish peroxidase to polymerize the monomer, yielding an ascorbic acid-functionalized polymer. Singh et al, “Biocatalytic Route to Ascorbic Acid-Modified polymers for Free Radical Scavenging” Advanced Materials, 15:1291-1294 (2003). The ascorbic acid, due to the regioselective enzymatic coupling process and mild reaction conditions, retained antioxidant activity based on free-radical scavenging.

Further advantages of enzyme-based polymer synthesis include, but are not limited to, environmental compatibility, selective reaction capability, and mild reaction conditions as compared to traditional off-chip synthetic routes.

A Peroxidases

Horseradish peroxidase (HRP) is a single-chain β-type hemoprotein that catalyzes the decomposition of hydrogen peroxide at the expense of aromatic proton donors. HRP is an Fe-containing porphyrin-type structure and is well-known to catalyze coupling of a number of phenol and aniline derivatives using hydrogen peroxide as oxidant. Likewise, soybean peroxidase may also polymerize monomers such as phenol. In one embodiment, HRP may catalyze the oxidative polymerization of cresol isomers and p-isopropylphenol.

Although it is not necessary to understand the mechanism of an invention, it is believed that the enzymatic polymerization may be used as an alternative for preparation of phenol polymers without using formaldehyde. Other advantages for enzymatic synthesis of useful polyphenols includes, but are not limited to: i) the polymerization of phenols under mild reaction conditions without use of toxic reagents (environmentally benign process); ii) phenol monomers having various substituents are polymerized to give a new class of functional polyaromatics; iii) the structure and solubility of the polymer can be controlled by changing the reaction conditions; and iv) the procedures of the polymerization as well as the polymer isolation are very facile.

In some embodiments, HRP catalyze aqueous polymerizations of hydrophobic monomers including, but not limited to, N-(4-hydroxyphenyl)maleimide, 4′-hydroxymethacrylanilide, and N-methacryloyl-1-aminoundecanoyl-4-hydroxyanilide in the presence of 2,6-di-O-methylated β-cyclodextrin.

In one embodiment, 4-hydroxyphenyl β-D-glucopyranoside (arbutin), undergoes a regioselective oxidative polymerization using a peroxidase catalyst in a buffer solution, yielding a water-soluble polymer consisting of a 2,6-phenylene unit. Similarly, a soybean peroxidase catalyzes the polymerization of 4-hydroxyphenyl benzoate.

In one embodiment, a photoactive azopolymer, poly(4-phenylazophenol), may be synthesized using an HRP enzyme. The polymer exhibits a reversible trans to cis photoisomerization of the azobenzene group with a long relaxation time.

In one embodiment, hydroquinone mono-oligo(ethylene glycol) ether may be polymerized by HRP in aqueous 1,4-dioxane.

In one embodiment, lignin (i.e., for example, a phenolic biopolymer) may be polymerized by an HRP-catalyzed terpolymerization of p-coumaryl alcohol, coniferyl alcohol, and sinapyl alcohol (14:80:6 mol %) in extremely dilute aqueous solutions at pH 5.5.

In one embodiment, lignin-degrading manganese(II) peroxidase may oxidatively polymerize various phenol derivatives including, but not limited to, guaiacol, o-cresol, and 2,6-dimethoxyphenol.

In some embodiments, peroxidases may catalyze the polymerization of anilines including, but not limited to, p-aminobenzoic acid, p-aminophenylmethylcarbitol, 2,5-diaminobenzenesulfonate, and p-aminochalcones.

In one embodiment, an HRP-catalyzed polymerization of o-phenylenediamine in an aqueous 1,4-dioxane provides a soluble polymer comprising an iminophenylene unit.

In one embodiment, an HRP-catalyzed polymerization of 4,4′-diaminoazobenzene provides a photodynamic polyaniline derivative containing an azo group.

In some embodiments, an HRP, hydrogen peroxide, and a β-diketone (i.e., for example, acetylactone) in a mixture of water and tetrahydrofuran may polymerize monomers including, but not limited to, hydrophobic monomers, styrene, and methyl methacrylate.

B. Laccases

Laccases having a Cu active site can be used to catalyze the oxidative coupling of phenols. In one embodiment, laccases may be derived from Pycnoporus coccineus (PCL) and Myceliophthore may polymerize syringic acid to give poly(oxy-2,6-dimethyl-1,4-phenylene)(poly(phenyleneoxide) (PPO). In another embodiment, enzymatic synthesis of PPO may also be achieved from 2,6-dimethylphenol using a PCL catalyst.

In one embodiment, the polymerization of 1-naphthol using laccase from Trametes versicolor (TVL) may proceed in aqueous acetone.

In one embodiment, coniferyl alcohol may be polymerized by a laccase catalyst.

In one embodiment, a laccase may polymerize acrylamide in water.

C. Oxidoreductases

In one embodiment, a copper-containing oxidoreductase (i.e., for example, bilirubin oxidase) may catalyze the oxidative polymerization of aniline and 1,5-dihydroxynaphthalene.

In some embodiments, a copper-containing monooxygenation enzyme (i.e., for example, a polyphenol oxidase such as tyrosinase) may be used as a catalyst for the modification of natural polymers. For example, a phenol moiety-incorporated chitosan derivative may be subjected to tyrosinase-catalyzed cross-linking, yielding a stable and self-sustaining gel. Alternatively, tyrosinase may also catalyze the hybrid production between a modified chitosan and protein.

In one embodiment, a water-resistant adhesive polymer may be produced by a tyrosinase-catalyzed reaction of 3,4-dihydroxyphenethylamine and chitosan.

In one embodiment, tyrosinase may catalyze the oxidative polymerization of soluble lignin fragments.

In one embodiment, glucose oxidase may catalyze vinyl polymerization in the presence of Fe²⁺ and dissolved oxygen.

D. Glycosyltransferases

In one embodiment, amylose may be polymerized using D-glucosyl phosphate as a substrate monomer and malto-oligomers catalyzed by a potato phosphorylase.

In one embodiment, poly(dimethylsiloxane-graft-R(D)-glucopyranose) may be polymerized using D-glucosyl phosphate, polysiloxane, maltoheptaonamide, and maltoheptaoside catalyzed by a potato phosphorylase. This method may also be used to produce polymers including, but not limited to, styryl-type amylose macromonomers, amylose-graft poly(L-glutamic acid), amylose-block-polystyrene, amylose-block-poly(ethylene oxide), and amylose containing silica gel.

In one embodiment, a cello-oligosaccharides polymer may be produced by a cello-oligosaccharide phosphorylase in the presence of a cellobiosyl and R-D-glucopyranosyl phosphate.

In one embodiment, cellulose and/or chitin may be polymerized from activated monomers including, but not limited to, uridine diphosphate glucose (UDPGlc), and UDP-N-acetyl-glucosamine (UDP-GlcNAc) catalyzed by cellulose and/or chitin synthases.

In one embodiment, a hyaluronic acid polymer may be produced using UDP-GlcNAc and UDP-glucronic acid (UDP-GlcA) catalyzed by a hyaluronic acid synthase. In one embodiment, the hyaluronic synthase may be selected from the group including, but not limited to, hyaluronic acid synthase, UDP-Glc dehydrogenase, UDP-Glc pyrophosphorylase, UDP-GlcNAc pyrophosphorylase, pyruvate kinase, lactate dehyrogenase, and inorganic pyrophosphatase.

In one embodiment, a chitin oligosaccharide may be polymerized by a chitin oligosaccharide synthase (NodC) using UDP-GlcNAc.

E. Acyltransferases

In one embodiment, a poly(hydroxyalkanoate (PHA) polymerase (i.e., for example, isolated from Ralstonia eutropha) may polymerize CoA monomers of (R)-hydroxyalkanoate into high molecular weight homopolymers and copolymers.

In one embodiment, a recombinant PHA synthase (i.e., for example, isolated from Chromatium vinosum) and a propionyl-CoA transferase (i.e., for example, isolated from Clostridium propionicum), may be used to polymerize (R)-hydroxybutyric acid into a poly(hydroxybutyrate) polymer.

F. Glycosidases

In one embodiment, a hexa-N-acetylchitohexaose and/or a hepta-N-acetylchitoheptaose may be polymerized from di-N-acetylchitobiose under the conditions of high substrate concentration (i.e., for example, 10 wt %), high ionic strength (i.e., for example, 30 wt % anunonium sulfate), and high temperature (i.e., for example, 70° C.) using an exo-glycosidase (i.e., for example, an egg yolk lysozyme).

In one embodiment, cellulase catalyzes the polycondensation of β-D-cellobiosyl fluoride to create artificial cellulose.

In one embodiment, xylan may be polymerized by a combination of cellulase and xylanase using β-xylo-biosyl fluoride as a substrate monomer in acetonitrile and acetate buffer.

In one embodiment, an artificial amylose (i.e., for example, a maltooligosaccharides) can be polymerized by polycondensation of R-D-maltosyl fluoride catalyzed by R-amylase in a methanol-phosphate buffer (pH 7).

In one embodiment, chitin may be enzymatically polymerized using chitinase and a chitobiose oxazoline derivative.

In one embodiment, a 6-O-Methyl-β-cellobiosyl fluoride may be polymerized by a cellulase to produce a 6-O-methylated cellulose polymer.

G. Lipases

In one embodiment, a nonsubstituted lactone (i.e., for example, having a ring size from 4 to 17) may be polymerized by a lipase catalyst to give a corresponding polyester. In one embodiment, a lipase catalyst may be a Candida rugosa lipase (lipase CR). In one embodiment, a lactone may be a β-propiolactone (β-PL, four-membered ring).

In one embodiment, PHB may be enzymatically polymerized from β-butyrolactone using either porcine pancreas lipase (PPL) or lipase CR.

In one embodiment, a racemic R-methyl-β-propiolactone may be stercoselectively polymerized by a lipase (i.e. for example, Pseudomonas cepacia lipase; lipase PC) to produce an optically active (S)-enriched polyester with an enantiomeric excess (ee) of approximately 50%.

In one embodiment, from a racemic β-BL, (R)-enriched PHB with 20-37% ee was formed by using thermophilic lipase as catalyst.

In one embodiment, a biodegradable poly(malic acid) may be produced by a lipase-catalyzed polymerization of benzyl β-malolactonate followed by debenzylation.

In some embodiments, polymer formation from γ-butyrolactone (γ-BL, a five membered ring) may be achieved by using PPL or Pseudomonas sp. lipase as catalyst.

In one embodiment, β-valerolactone (β-VL, a six-membered ring) may be polymerized by a plurality of lipases.

In one embodiment, 1,4-dioxan-2-one (a six-membered lactone) may be polymerized by Candida antarctica lipase (lipase CA)

In one embodiment, lipase CA may polymerize α-methyl-substituted medium-size lactones including, but not limited to, α-methyl-δ-valerolactone (six-membered ring) and α-methyl-ε-caprolactone (seven-membered ring).

In one embodiment, lipase PC may catalyze an enantioselective polymerization of 3-methyl-4-oxa-6-hexanolide (a seven-membered ring).

In some embodiment, lipases may polymerize large lactone ring structures including, but not limited to, 8-octanolide (a nine-membered ring) and racemic fluorinated lactones having a ring size ranging from 10-14 to create optically active polyesters.

In some embodiments, macrolide polymers may be produced using macrolide monomers including, but not limited to, 11-undecanolide (a 12-membered ring; UDL), 12-dodecanolide (a 13-membered ring; DDL), 15-pentadecanolide (a 16-membered ring; PDL), and 16-hexadecanolide (a 17-membered ring; HDL) catalyzed by a lipase.

In one embodiment, polyesters bearing a sugar moiety at the polymer terminal may be polymerized by lipase CA-catalyzed polymerization of ε-caprolactone (ε-CL, seven-membered lactone) in the presence of an alkyl glucopyranoside and/or a polysaccharide.

In one embodiment, polyester macromonomers and/or telechelics may be produced by a lipase-catalyzed polymerization of DDL using vinyl esters as a terminator. In one embodiment, the terminator comprises vinyl methacrylate and the lipase comprises Pseudomonas fluorescens lipase (lipase PF) and a methacryl-type macromonomer can be produced.

In one embodiment, a lactide may be polymerized using lipase PC at a temperature ranging between approximately 80-130° C. to produce a poly(lactic acid).

In some embodiments, lipases CA, PC, and/or PF may catalyze the polymerization of ethylene dodecanoate and ethylene tridecanoate to produce their corresponding polyesters.

In one embodiment, trimethylene carbonate (a six membered ring; TMC) may be polymerized by lipases including, but not limited to, CA, PC, PF, and PPL to produce their corresponding polycarbonate.

In one embodiment, a water-soluble polycarbonate having pendent carboxyl groups on the polymer main chain may be produced by a lipase-catalyzed polymerization of 5-methyl-5-benzyloxycarbonyl-1,3-dioxan-2-one, followed by debenzylation.

In some embodiments, cyclic dicarbonates, including, but not limited to, cyclobis(hexamethylene carbonate) and cyclobis(diethylene glycol carbonate) may be polymerized by lipase CA.

In one embodiment, a random ester-carbonate copolymer may be enzymatically polymerized by a lipase using DDL-cyclobis(diethylene glycol carbonate) and lactide-TMC.

In one embodiment, a dicarboxylic polyester may be polymerized using a lipase-catalyzed polycondensation of adipic acid and 1,4-butanediol and/or 1,6-hexanediol.

In one embodiment, an aliphatic polyester may be polymerized using diacids and glycols in a solvent-free system catalyzed by lipase CA.

In some embodiments, lipases including, but not limited to, lipases CA, CC, and Mucor miehei lipase (lipase MM) may catalyze the polymerization of sebacic acid and 1,8-octanediol.

In one embodiment, lipase CA or lipase MM may catalyze the polycondensation of dimethyl succinate and 1,6-hexanediol in toluene.

In some embodiment, lipase may polymerize halogenated alcohols, including, but not limited to, 2-chloroethanol, 2,2,2-trifluoroethanol, and 2,2,2-trichloroethanol by catalyzing esterifications and/or transesterifications.

In some embodiments, ester copolymers may be synthesized by lipase catalyzed copolymerization of lactones, divinyl esters, or glycols.

In one embodiment, a polyester may be created by the polymerization of succinic anhydride with 1,8-octanediol using a lipase PF at room temperature.

In one embodiment, a polyester may be created by the polymerization of poly(azelaic anhydride) and a glycol using a lipase CA catalyst.

In one embodiment, an oxirane polymer may be created using succinic anhydride and a PPL catalyst.

In one embodiment, polyesters containing an aromatic moiety in the main chain may be created from divinyl esters of isophthalic acid, terephthalic acid, and p-phenylene diacetic acid with a glycol by lipase CA.

In one embodiment, an optically pure polyester may be synthesized by PPL-catalyzed enantioselective polymerization of bis(2,2,2-trichloroethyl)trans-3,4-epoxyadipate with 1,4-butanediol in diethyl ether.

In one embodiment, lipase CA-catalyzed regioselective polymerization of divinyl sebacate and glycerol may produce divinyl esters and polyols.

In one embodiment, the lipase CA-catalyzed polymerization of divinyl esters and sorbitol regioselectively produces a sugar-containing polyester with the 1,6-diacylated unit of sorbitol. Alternatively, mannitol or meso-erythritol may also be regioselectively polymerized from divinyl sebacate.

In one embodiment, sugar diesters may be produced by lipase CA-catalyzed esterification of sucrose or trehalose with divinyl adipate in acetone, in which the 6- and 6′-positions of the starting sugar were regioselectively acylated. Lipase CA may also catalyze the subsequent polycondensation of the isolated diesters with glycols to give sugar-containing polyesters.

In one embodiment, lipase CA-catalyzed polymerization of dimethylmaleate and 1,6-hexanediol may be performed in toluene.

In one embodiment, dimethyl maleate and dimethyl fumarate with 1,6-hexanediol may be copolymerized using lipase CA.

In one embodiment, PPL may catalyze the transesterification of triglycerides in an excess of 1,4-cyclohexanedimethanol to produce 2-monoglycerides.

In one embodiment, fluorinated polyesters were synthesized by the enzymatic polymerization of divinyl adipate with fluorinated diols (i.e., for example, 3,3,4,4,5,5,6,6-octafluorooctan-1,8-diol) using lipase CA.

In one embodiment, polycarbonate synthesis using lipase-catalyzed polycondensation comprises activated dicarbonate and 1,3-propanediol divinyl dicarbonate.

In one embodiment, lipase CA may polymerize an α,ω-alkylene glycol to produce a polycarbonate with a molecular weight up to 8.5×10³.

In one embodiment, high molecular weight polycarbonates may be enzymatically polymerized using diethyl carbonate, 1,3-propanediol and/or 1,4-butanediol.

In one embodiment, PPL may catalyze the polymerization of 12-hydroxydodenacoid acid.

In one embodiment, PPL may catalyze the polymerization of methyl esters of 5-hydroxypentanoic and/or 6-hydroxyhexanoic acids in hexane.

In some embodiments, a Pseudomonas sp. lipase may catalyze the polymerization of ethyl esters of 3- and 4-hydroxybutyric acids, 5- and 6-hydroxyhexanoic acids, 5-hydroxydodecanoic acid, or 15-hydroxypentadecanoic acid.

In one embodiment, optically active polyesters may be enzymatically polymerized using oxyacid derivatives. For example, in a lipase CR catalyzed polymerization of racemic 10-hydroxyundecanoic acid, the resulting oligomer was enriched in the (S)-enantiomer.

H. Proteases

In one embodiment, proteases including, but not limited to, papain or R-chymotrypsin may catalyze the polymerization of diethyl L-glutamate hydrochloride to produce a polymer comprising at least one R-peptide linkage.

In one embodiment, diethyl L-aspartate may be polymerized using alkanophilic protease (i.e., for example, isolated from Streptomyces sp.) to produce a polymer comprising α- and β-peptide linkages.

In one embodiment, protease mutants may show a higher catalytic activity for the enzymatic polymerization of amino acid esters in an aqueous DMF solution when compared to a wild type protease. For example, a subtilisin mutant (subtilisin 8350) derived from BPN′ (subtilisin from Bacillus amyloliquefaciens) via six site-specific mutants (Met 50 Phe, Gly 169 Ala, Asn 76 Asp, Gln 206 Cys, Tyr 217 Lys, and Asp 218 Ser) in the polymerization of L-methionine methyl ester in the aqueous DMF. Alternatively, another mutant (subtilisin 8397), which is the same as subtilisin 8350 without changing Tyr 217, induced the polymerization of single amino acid, dipeptide, and tripeptide methyl esters.

In one embodiment, a peptide hydrolase (i.e., for example, dipeptide transferase) may catalyze the polymerization of a dipeptide amide (i.e., for example, glycyl-L-tyrosinamide).

In one embodiment, a polycondensation of sucrose with bis(2,2,2-trifluoroethyl)adipate using an alkaline protease (i.e., for example, isolated from Bacillus sp.) as catalyst regioselectively created an oligoester having ester linkages at the 6- and 1′-positions on the sucrose.

I. Hydrolases

In one embodiment, a PHB depolymerase catalyzes the ring-opening polymerization of cyclic monomers. For example, a PHB depolymerase (i.e., for example, isolated from Pseudomonas stuizeri YM1006) polymerized β-BL.

In one embodiment, ε-CL and TMC may be polymerized by PHB depolymerase (i.e., for example, isolated from Pseudomonas lemoignei).

V. Polymerization on a Chip

In one embodiment, the present invention contemplates a microchip device capable of performing an enzymatic analysis. In one embodiment, the enzymatic analysis comprises a microenzymatic polymerization resulting in catalytic biomaterial synthesis. This capability represents a significant improvement over previous techniques limited to enzymatic assays and enzymatic reaction related analysis. Gross et al., “Enzymes in polymer synthesis” American Chemical Society Symposium 684 (1996); Cross et al., “Process Intensification—Laminar-Flow Heat-Transfer,” Chemical Engineering Research & Design, 64:293-301 (1986); Maner et al., “Mass-Production of Microdevices with Extreme Aspect Ratios by Electroforming” Plating and Surface Finishing, 75:60-65 (1988); Hagmann et al., “Fabrication of Microstructures of Extreme Structural Heights by Reaction Injection-Molding” International Polymer Processing, 4:188-195 (1989); Jacobson et al, “High-Speed Separations on a Microchip,” Analytical Chemistry, 66:1114-1118 (1994); Hessel et al., “Potentials and Realization of Microreactors,” International Symposium on Microsystems, Intelligent Materials and Robots, Sendai, Japan (1995); Kovacs G., In: Micromachined Transducers Sourcebook, Boston: WCB/McGraw-Hill (1998); Madou M., In: Fundamentals of Microfabrication, New York: CRC Press (1997); Ayon et al., “Characterization of a Time Multiplexed Inductively Coupled Plasma Etcher,” J Electrochemical Society, 146:339-349 (1999); Ayon et al., “Influence of Coil Power on the Etching Characteristics in a High Density Plasma Etcher,” J Electrochemical Society, 146:2730 (1999); Kovacs G., In: Micromachined Transducers Sourcebook. New York: McGraw-Hill (1998); Petersen K., “Silicon as a mechanical material,” Proc. IEEE, 70:420-457 (1982); Jo et al., “Three-dimensional micro-channel fabrication in polydimethylsiloxane(PDMS) elastomer,” J. Microelectromech. Syst., 9:76-81 (2000); Becker et al., “Polymer microfabrication methods for microfluidic analytical applications,” Electrophoresis, 21:12-26 (2000); Xu et al., “Room-temperature imprinting method for plastic microchannel fabrication,” Anal. Chem, 72:1930-1933 (2000); McCormick et al., “Microchannel electrophoretic separations of DNA in injection-molded plastic substrates” Anal Chem. 69:2626-2630 (1997); Hooper H., “Microchannel electrophoretic separations of DNA in injection-molded plastic substrates” Anal. Chem, 69: 2626-2630 (1997); Roberts et al., “UV laser machined polymer substrates for the development of microdiagnostic systems” Anal. Chem., 69:2035-2042 (1997); Weigl et al., “Design and rapid prototyping of thin-film laminate-based microfluidic devices” Biomed. Microdevices, 3:267-274 (2001); Johnson et al., “Microfabricated structures for DNA analysis” Proc. Natl. Acad. Sci. USA, 93:5556-5561 (1996); and Selvaganaphthy et al., “Recent Progress in Microfluidic devices for Nucleic Acid and Antibody Assays” IEEE, 91(6) (2003).

Currently, most microenzymatic research involves on-chip enzymatic assay technology. On-chip enzymatic assays belong to the class of microchip derivatization protocols in which a non-detectable specie is converted to a detectable one. Wong J., “On-chip enzymatic assays” Electrophoresis, 23:713-718 (2002). Enzyme assays are not useful for all applications related to enzyme research. For example, commercially available enzyme-based microchips are limited to on-chip assays of substrates, on-chip assays of inhibitors, on-column reactions, post-column reactions, and microchips based on immobilized enzymes, as well as other chip-based enzymatic devices. Lee et al., “Micro total analysis system in biotechnology” Appl. Microbiol Biotechnol. 64:289-299 (2004). Generally, the enzyme assay is a measurement of a chemical reaction, which might involve measuring the formation of the product.

While early microchip derivatization or separation assays have focused on chemical reactions, the use of enzymes can impart high selectivity into microchip devices, and expand their scope towards analytically important substrates. In addition to assays of substrates, an enzyme-based microchip device offers convenient determination of enzyme inhibitors, measurements of enzyme activities, or for identifying enzymes among separated components. Such assays usually rely on on-chip mixing and reactions of the molecular substrate and enzymes in connection to separations of the substrates or products. Although it is not necessary to understand the mechanism of an invention, it is believed that performing on-chip enzymatic reactions requires an understanding of how enzymatic reactions behave on a small scale, integration with separation microchips, and how microfluidics can be tailored to suit the requirements of particular enzymatic analysis.

It is further believed that enzymatic microdevices as contemplated herein, improve reaction times without compromising the quality of the analytical separation, save resources, improve precision, and improve safety. The versatility of such on-chip enzymatic chips offer great promise for decentralized testing of clinically or environmentally important molecular substrate. For example, enzyme cascades can specifically convert 100-100,000 substrate molecules to a final product within 10 seconds. Enzymes comprising catalytic and selectivity properties will find widespread use in chemical analysis, including conventional flow-injection analysis and traditional capillary electrophoresis systems. One illustration provided herein describes a simultaneous on-chip generation of an ascorbic acid conjugated monomer followed by a polymerization using microscale enzymatic selection methods. The versatility of such enzymatic microchips offer great promise for decentralized testing of clinically or environmentally important substrates.

In one embodiment, a microscale enzymatic cascade chip polymerizes functional biomaterials using enzymatic catalysis reactions. In one embodiment, the chip comprises a disposable multi-step monolithic device with multi-fluidic manipulating functions. In one embodiment, the chip performs a simultaneous on-chip synthesis of ascorbic acid polymerization with highly enzymatic selection methods. In one embodiment, the enzymatic catalytic reactions comprise oxidase and dehydrogenase. In another embodiment, the enzymatic catalytic reactions synthesize VC polymers using nine (9) chemicals and a two (2) stage reaction. In one embodiment, the catalytic reactions occur without micromachining (i.e., without any moving parts) wherein a mixing, reacting, separating process occurs by sequential on-chip transportation. In one embodiment, separating a final product by molecular size comprises a 20 μm comb filter array embedded in the micropanning wells. In another embodiment, the microscale enzymatic cascade chip provides for precise temperature control.

### VI. Antioxidant Compositions

A variety of products and properties are contemplated including, but not limited to, antioxidant compositions. In one embodiment, the present invention contemplates an anti-aging composition comprising a polymer and an antioxidant. In one embodiment, the composition comprises a polymerized biomaterial functionalized with antioxidants. In one embodiment, the composition comprises controlled release of an antioxidant stabilized pharmaceutical to provide improved treatments. In one embodiment, the present invention contemplates a method for treating free-radical induced aging phenomena using a composition comprising a polymer and an antioxidant.

In one embodiment, polymerized biomaterial functioning as ‘green’ antioxidants could prove useful in therapeutic treatments to reduce and/or eliminate free-radical induced aging phenomena, or to stabilize controlled release of pharmaceuticals. In one embodiment, ascorbic acid conjugated polymers could provide more stable pharmaceuticals.

As detailed above, these antioxidant polymers could find use in many applications where free-radical scavenging is desired. The enzymatic polymers described herein could improve the shelf-life of labile components in foods while also reducing human exposure to freely soluble antioxidants such as BHT and BHA. Antioxidant benefits could also be conferred on personal care products leading to reduced human exposure to these compounds. Biomaterials functionalized with antioxidants could also prove useful in therapeutic treatments to reduce and/or eliminate free-radical induced aging phenomena, or for controlled release of pharmaceuticals where better control of ascorbic acid content could provide improved treatments. In general, polymer backbones having an ascorbic acid antioxidant moiety has implications for many consumer-related applications including, but not limited to, foods, pharmaceuticals, and personal care products. See Table 6.

TABLE 6
Consumer-Related Applications For Enzymatic Polymerization Products

| | | | Biomimetrics |
|---|---|---|---|
| | | | (altered physical |
| Consumer Products | Green Chemistry | Medical Products | properties) |
| Food Preparation. | Green Polymerization | Anti-aging products | Synthesis and |
| Detergent Additives | for pharmaceuticals | (i.e., for example, | degradation of |
| (i.e., for example, | and agrochemicals. | ascorbic acid | proteins, polyphenols, |
| lipases and proteases). | | polymers). | etc. to modify protein |
| | | | functionality. |
| Flavor Enhancers. | Low cost enzymatic | Nontoxic natural | Non-protein based |
| Evaluating Food | polymers for food | catalyst with | catalytic systems, e.g., |
| Processing-Induced | packaging research | ecological | ribozymes. |
| Changes, | and development. | requirement. | |
| Food Dextrin. | Green chemical lab on | Condensation, ring | Amylase applications. |
| Sugar Products. | a chip, mild reaction | opening | |
| Syrup Manufacture. | catalysis. | polymerization. | |
| Starch Removal (i.e., | High enatio-, regio-, | Dextrose | |
| for example, from | and chemoselectivies | manufacture. | |
| fruit juice) | as well as regulation | Dry breakfast cereals. | |
| Dextrose | of stereochemistry | | |
| Manufacture. | providing | | |
| | development of new | | |
| | reactions to function | | |
| | compounds. | | |

### EXPERIMENTAL

### Example 1

### On-Chip Polymerization of Poly L-Ascorbyl Methyl Methacrylate

This example illustrates one embodiment of a microfluidic microchip polymerization by an enzymatic cascade.

Tables I and II provide the step-by-step procedure that was used to synthesize P-AA-MMA.

TABLE 1
Stage I Enzymatic Transesterification: Synthesis
Of L-Ascorbyl Methyl Methacrylate

| Step (Reaction Vessel 1) | Material | Quantity Used | |
|---|---|---|---|
| Substrate (G1.1) | L-ascorbic acid | 150 | mg |
| | (AA) + 50% AD | 0.852 | mM |
| | 2,2,2-trifluoroethyl | 0.182 | ml |
| | methacrylate | 1.278 | mM |
| Enzyme (G1.2) | Anhydrous Dioxane | 1.5 | ml × 2 |
| | Antartica Lipase | 12.5 | mg |
| Anti-poly @ 60° C. (G1.3) | 2,6-di-tert-butyl-4- | 2.5 | mg |
| | methylphenol | | |

Reaction Temperature: 50-60° C. Reaction Time: 45-60 minutes. Flow Rate <0.01 ml/minute

TABLE 2
Stage II HRP Polymerization: Synthesis
Of Poly L-Ascorbyl Methyl Methacrylate

| Step (Reaction Vessel 1) | Material | Quantity Used | |
|---|---|---|---|
| Mix 1 (G2.1 w/G2.2) | L-Ascorbyl methyl | 0.02 | g |
| | methacrylate | 0.082 | mM |
| | Tetrahydrofuran (THF: | 0.11 | ml |
| | solvent) N₂ Flushed. | | |
| | HRP w./water 15 minutes | 1.6 mg/0.05 ml | |
| Dissolve (G2.2) | Water | 2 | μl |
| | Hydrogen Peroxide | 9.3 | μl |
| Mix 2 (G2.3) 2 hours | 2,4-pentanedione | 1.77 | μl |
| | (polymerization catalyst) | | |

Reaction Time: 60-90 minutes. Shaking Time: 20-30 minutes. Flow Rate <0.01 ml/minute

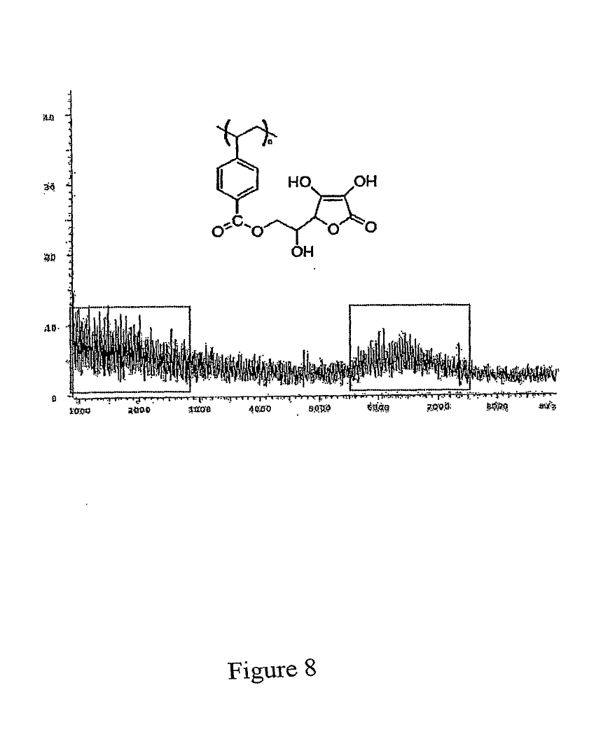

The on-chip polymerization of P-AA-MMA utilized highly enzymatic selection methods, while the enzyme detection included conventional methods of detecting ascorbic acid. For example, the polymerization is based on reactions between an oxidase and dehydrogenase enzymes. This strategy resulting in the covalent coupling of the primary hydroxyl group of ascorbic acid with a monomer. Second, a horseradish peroxidase (HRP) was used to polymerize the functional monomer, yielding an ascorbic acid-functionalized polymer. See FIG. 8.

The separation of the final product, with a much larger polymerized molecular size and weight, from the mixture has been achieved by using 20 μm comb filter array embedded in the micro-panning wells. In general, this can be described as enzymatic-catalytic reaction and polymerization. The exploration of microscale polymerized ascorbic acid is exciting and new, which make microscale ascorbic acid polymerization chip design and development novel in the microchip development domain.

### Example II

### Antioxidant Effect of an Ascorbic Acid-Functionalized Vinyl Polymer

This example describes the synthesis of and antioxidant testing of a polymer-ascorbic acid conjugate.

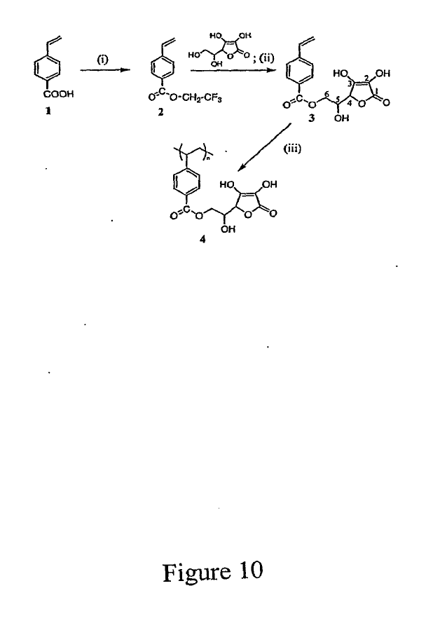

Immobilized Candida antarctica lipase and L-ascorbic acid were dried under high vacuum in a desiccator with phosphorous pentoxide for 24 h prior to reaction. The reaction approach was an enzymatic transesterification where the primary hydroxyl group of ascorbic acid was regioselectively acylated by trifluoroethyl 4-vinylbenzoate via the acyl enzyme complex. (See FIG. 10).

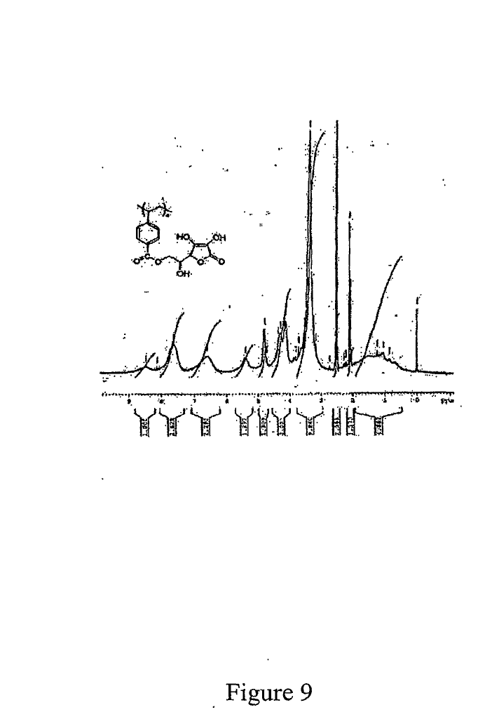

In the ¹H NMR spectrum of L-ascorbyl-vinyl benzoate, C-6H (methylene protons) appeared at δ 4.47, which otherwise appeared at δ 3.68 in ascorbic acid. (See FIG. 9) This downfield shift indicated the formation of an ester involving the C-6-OH group. In addition, the integral ratio of vinyl protons and ascorbic acid corresponded to a monoacylated product. In a ¹³C NMR spectrum of L-ascorbyl 4-vinylbenzoate, the C-6, methylene carbon appeared at δ 77.45 which otherwise appeared at δ 63.60 in ascorbic acid, indicating ester formation with C-6-0. No significant shift was observed in the C-2, C-3, or C-5 carbons. (data not shown) Furthermore, the study of integral values of ¹H NMR signals, as well as peak positions in proton and carbon NMR, confirmed that the expected product was formed.

Horseradish peroxidase catalyzed polymerization of L-ascorbyl-4-vinylbenzoate was carried out with oxidant hydrogen peroxide and initiator 2,4-pentane-dione. 2,4-Pentanedione was distilled under vacuum before use. In a general procedure of polymerization of L-ascorbyl-4-vinylbenzoate, 1.8 mL water and 2.0 mL methanol were flushed with nitrogen for 10 min. L-Ascorbyl-4-vinylbenzoate (457 mg, 1.5 mM) was added to the reaction mixture. Horseradish peroxidase (16 mg) was dissolved in 200 μl of water. Hydrogen peroxide, 0.15 mM (17 μl) and 0.30 mM of 2,4-pentanedione were added simultaneously after the addition of the enzyme. Polymerization was conducted for 24 h with continuous stirring. The crude product was washed with acetone to remove non-polymerized monomer. The product was dried under vacuum and analyzed by ¹H NMR and matrix-assisted laser desorption-ionization time-of-flight mass spectrometry (MALDI-TOF MS). The ¹H NMR spectrum of Compound 4 showed the presence of methylene and methine protons as broad peak from δ 0.85 to 2.75 and an absence of vinyl protons at δ 5.39, 5.93, and 6.80, indicating successful vinyl polymerization. (See FIG. 9).

Aromatic protons appear as broad singlets at δ 6.60 and 7.63. Furthermore, the presence of C-5 and C-6 protons at δ 4.13 and 4.63 confirmed that the vinyl group was polymerized and ascorbic acid was attached as pendent group through a C-6-0 linkage. MALDI-TOP MS of methanol soluble fractions showed a polymer with number-average molecular weight (M=1225 and polydispersity (PD)=1.03. For polymer fractions soluble in dimethylsulfoxide, MALDI-TOF peaks up to 7000 were detected. Higher molecular weight in soluble polymer could not be analyzed.

Ascorbic acid when used at a concentration up to 187 μM fully scavenged 2,2-diphenyl-1-picryl hydrazyl (DPPH) free radicals (0.2 mM), while compound 4 was able to fully scavenge free radicals when used at a concentration as low as 238 μM. (See Table 3).

TABLE 3
Free Radical Scavenging Effect Of L-ascrobyl-4-vinylbenzoate
(4) on 0.2 mM DPPH

| | | Concentration | Absorbance |
|---|---|---|---|
| Rx. No | Compound | [μm] | at λ = 514 nm [a] |
| 1 | DPPH (blank) | 200 | 1.12 ± 0.01 |
| 2 | Ascorbic acid | 329 | [b] |
| 3 | Ascorbic acid | 187 | [b] |
| 4 | Ascorbic acid | 91 | 0.12 ± 0.01 |
| 5 | Polymer (4) | 663 | [b] |
| 6 | Polymer (4) | 330 | [b] |
| 7 | Polymer (4) | 238 | [b] |
| 8 | Polymer (4) | 189 | 1.0 ± 0.0 |
| 9 | Polymer (4) | 132 | 1.39 ± 0.01 |
| 10 | Polymer (4) | 91 | 1.67 ± 0.01 |
| 11 | Polymer (4) | 58 | 1.87 ± 0.01 |

[a] Reactions were performed in triplicate; average ± standard deviation.
[b] All DPPH scavenged by compound.

This analysis showed the formation of the vinyl polymer with active pendent antioxidant compounds since the ascorbic acid retained its ability to scavenge radicals while in polymeric form. Aromatic protons appear as broad singlets at ε 6.60 and 7.63. Furthermore the presence of C-5 and C-6 protons at δ 4.13 to 4.63 confirmed that the vinyl group was polymerized and ascorbic acid was attached as pendant group through a C-6-0 linkage.

### Example III

### A Microfluidic Device Having Patterned Stacked Channels

This example presents one method by which a microfluidic device may be fabricated comprising stacked channels.

A patterned silicon master was developed by DRIE of Si <100> with a pattern of reactors and channels. The depth of the channels was 90 μm and the lowest width was 10 μm. PDMS was cast onto the channels to create the patterned structures. PDMS was prepared in two different polymer to curing agent ratios (10:1, 12.5:1). The mixture was immediately placed in a vacuum for ˜30 minutes until all bubbles have been released. The PDMS was then cured for 1-2 hours at 60° C. The PDMS casting channels and Pyrex® slides steps were cleaned with 70% alcohol before preheating or ashing. For pre-heated samples, the PDMS and slide were kept in a thermal chamber at 65° C. for 30 minutes. The were then transferred to the plasma asher in under one minute. In the asher, the vacuum is pulled and then oxygen applied. The RF was then applied to create the plasma for approximately one minute. The PDMS was then adhered to the step first then to the substrate. The detached length is then measured.

For a 12.5:1 PDMS to curing agent ratio, there were three experiments with preheating and three with no preheat. Different step heights were investigated to analyze the results broadly since the method has a non-physical step height dependence. For the 10:1 PDMS to curing ratio, there were four experiments with preheat and four experiments with no preheat.

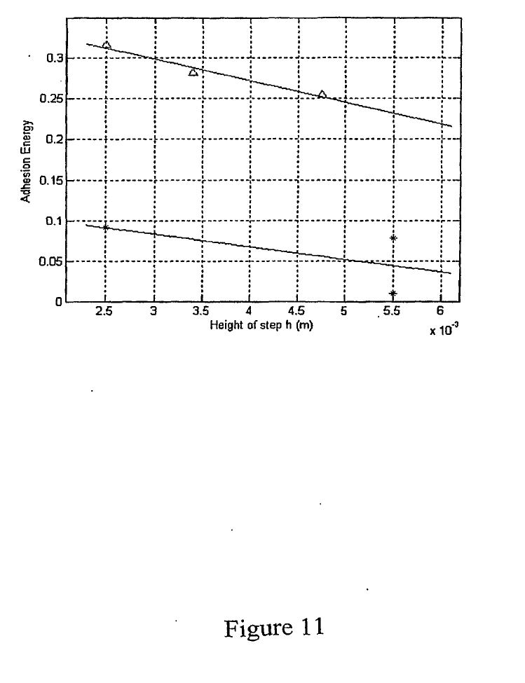

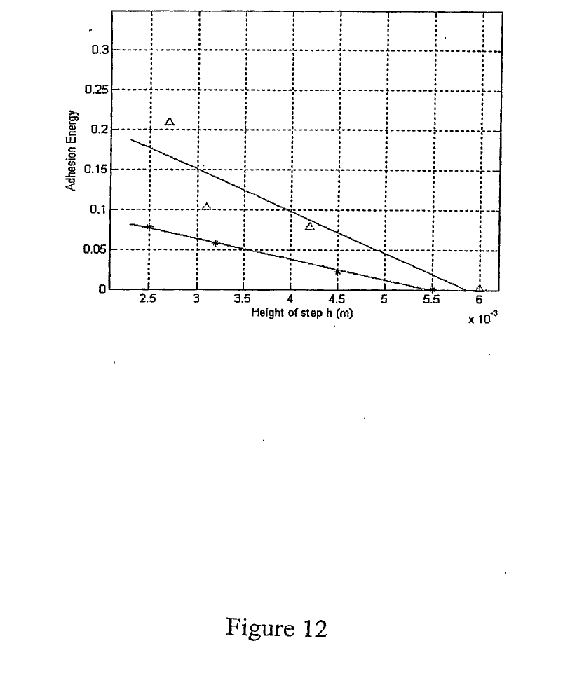

The calculated adhesion energy density for experiments are shown in FIG. 4 for the PDMS to curing agent ratio of 12.5:1 and for the 10:1 PDMS to curing agent ratio (See FIGS. 11 and 12, respectively).

For both mixing ratios, the preheated samples show significant increase in adhesion energy density. Comparisons between preheat and no-preheat are made at similar step heights. For the 12.5:1 mixing ratio, there is a consistent increase of 0.15 Jm⁻² for all height steps. For the 10:1 ratio the adhesion energy density increase ranges from 0.05 to 0.15 Jm⁻². At room temperature plasma processing, the mixing ratio has less of an effect on adhesion energy density.

The increase in adhesion energy density may be due to an increased amount of surface reaction during plasma oxidation above room temperature. One possible explanation is that the preheating process will stimulate more molecules at the surface which creates more hydroxyl groups. Another interpretation is that more hydrophilic groups per unit area do not revert back to a hydrophobic state at elevated temperatures. The results from the variation of mixing ratio indicates that the increase in adhesion energy density with preheat may be due to more availability of PDMS at the surface.

### Example IV

### Microcomb Filter Selection

This example presents several variations of microcomb filters that were tested in development of some embodiments of the microfluidic enzymatic cascade system contemplated herein.

The requirements for an appropriate filter included not only a capability for mixing and fluid manipulations but also sample purification. Consequently, a microcomb filter selection process was performed. See Table 5.

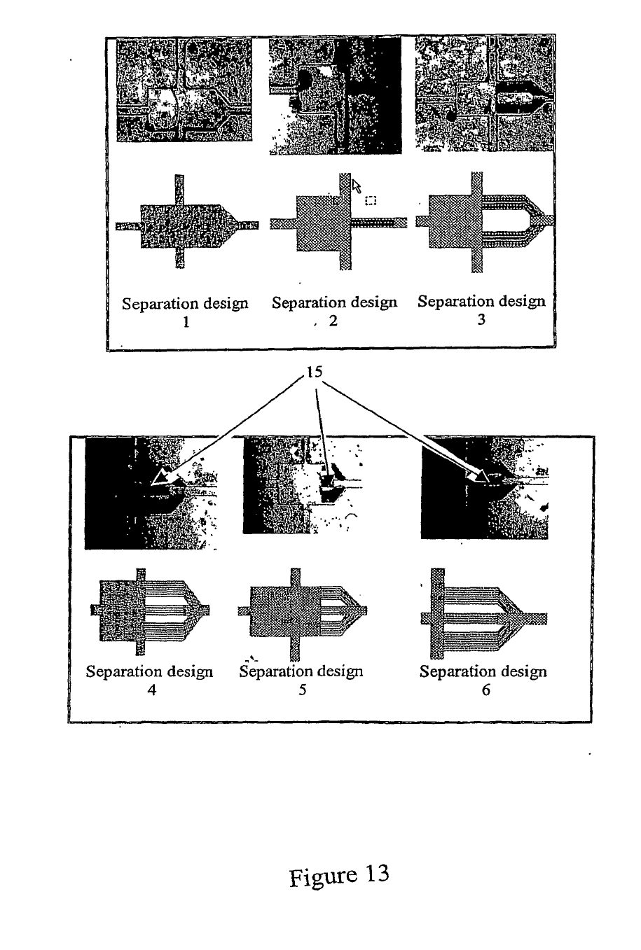

Six (6) separation microcomb designs variations were evaluated (See FIG. 13).

Design 1: having a reaction chamber (2) thereby not functioning as a channel separation filter.

Design 2: having a reaction chamber and a central microcomb filter (15) thereby functioning as a low pass separator.

Design 3: having a reaction chamber and two side microcomb filters (12), thereby functioning as a medium pass separator.

Design 4: having a reaction chamber, a central microcomb filter, and two side microcomb filters, thereby acting as a high pass separator.

Design 5: having a reaction chamber, a central microcomb filter, and two side microcomb filters, thereby acting as a medium pass separator.

Design 6: having a central microcomb filter and two side microcomb filters, thereby acting as a high pass separator.

TABLE 5
Comparison Of Reactor/Microcomb Filter Designs

| | Reaction | Filtering | | |
|---|---|---|---|---|
| Design | Area | Length | | |
| # | (mm²) | (mm) | Advantages | Disadvantages |
| 1 | 2.1 | 0 | Large Reaction Area | No Filtering |
| 2 | 1.0 | 2 | Medium Reaction | Limited |
| | | | Area | Filtering |
| 3 | 1.0 | 24.5 | Large Reaction Area | |
| | | | And Medium Filtering | |
| 4 | 1.0 | 26 | Large Reaction Area | |
| | | | And Long Filtering | |
| 5 | 1.5 | 24 | Large Reaction Area | |
| | | | And Medium Filtering | |
| 6 | 0.2 | 26 | Long Filtering | Small Reaction |
| | | | | Area |

Design 4 presents the most balanced design by combining the longest filtering length with an efficient reaction area.

### Example V

### Enzymatic Polymerization of Polyhydroxyalkanoates

This example illustrates one embodiment for a biosynthetic pathway to polymerize polyhydroxyalkanoates.

Polyhydroxyalkanoates (PHA) comprise a family of polyesters produced by microorganisms in nature. The material can made through PHA synthase producing a wide range of properties with some versions having properties similar to polypropylene—but are biodegradable.

Illustrated below is one biosynthesis pathway through a Condensation Reduction Reaction. Sudesh et al., “Polyhydroxyalkanoates,” In: Handbook of Biodegradable Polymers, Editor: Catia Bastioli, Rapra Technology Limited, pgs. 219-256 (2005). The three enzymes perform monomer activitation, reduction, and polymerization of Acetyl-CoA (developed from sugars) to PHA in sequence. (See flow chart below).

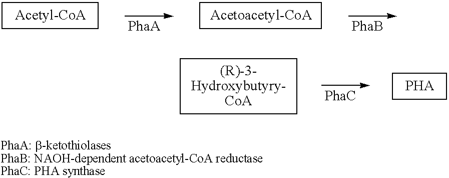

These enzymes will be integrated into the microfluidic devices contemplated herein to produce PHA.

### Example VI

### P Enzymatic Polymerization of Lactones

This example illustrates one embodiment for a biosynthetic pathway to polymerize polyhydroxyalkanoates. Kirpal et al., “Ethyl Glucoside as a Multifunctional Initiator for Enzyme-Catalyzed Regioselective Lactone Ring-Opening Polymerization,” J. Am. Chem. Soc., 120:1363-1367 (1998).

The one-pot biocatalytic synthesis of novel amphiphilic products consisting of an ethyl glucopyranoside (EGP) headgroup and a hydrophobic chain is described. The porcine pancreatic lipase (PPL) catalyzed ring-opening polymerization of ε-caprolactone (ε-CL) by the multifunctional initiator EGP was carried out at 70° C. in bulk. Products of variable oligo(ε-CL) chain length (Mₙ=450, 220) were formed by variation of the ε-CL/EGP ratio. Extension of this approach using Candida antarctica lipase (Novozym-435) and EGP as the initiator for trimethylene carbonate (TMC) ring-opening polymerization also resulted in the formation of an EGP-oligo(TMC) conjugate (Mₙ=7,200). Structural analysis by ¹H, ¹³C, and COSY (¹³C-¹³C) NMR experiments showed that the reaction was highly regiospecific; i.e., the oligo(ε-CL)/oligo(TMC) chains formed were attached by an ester/carbonate link exclusively to the primary hydroxyl moiety of EGP.

These enzymes will be integrated into the microfluidic devices contemplated herein to produce lactone polymers.

### Example VI

### Enzymatic Polymerization of Proteins

This example illustrates one embodiment for a biosynthetic pathway to polymerize proteins. Aruna Nathan et al, “Amino Acid Derived Polymers,” In: Biomedical Polymers. Designed-to-Degrade Systems, Ed. Shallaby W. Shalaby, Hansser Publishers, 1994, pp. 117-120.

Amino acids are conjugated with N-caroxyanhydride using a ring opening polymerization reaction with lipase to form poly(amino acids).

These enzymes will be integrated into the microfluidic devices contemplated herein to produce poly(amino acids).

We claim:

1. A method, comprising:
a) providing:
i) a substrate loading deck into which a sample comprising a first reactant is introduced;
ii) a plurality of reactant loading decks into which at least one additional reactants are introduced;
iii) a plurality of reaction chambers comprising at least one enzyme, wherein said chambers are in fluidic communication with said substrate loading deck and said reactant loading decks;
b) introducing said substrate into said substrate loading deck under conditions such that said substrate moves into a first reaction chamber;
c) introducing an additional reactant into a first reactant loading deck under conditions such that said second reactant moves into said first reaction chamber; and
d) reacting said sample and said additional reactant in said first reaction chamber under conditions such that a polymer is formed.

2. The method of claim 1, wherein said reaction chambers further comprise a microcomb filter for separating said polymer.

3. The method of claim 1, further comprising symmetrically branched microchannels fluidly connecting said sample loading deck, said reactant loading decks reaction chambers.

4. The method of claim 2, wherein said reaction chamber further comprises at least one side channel for collecting unreacted sample and unreacted additional reactants.

5. The method of claim 1, wherein said introducing comprises an injection.

6. The method of claim 2, wherein said microcomb filter comprises two side microcombs.

7. The method of claim 2, wherein said microcomb filter comprises a central microcomb and two side microcombs.

8. The method of claim 1, wherein said substrate comprises an antioxidant

9. The method of claim 1, wherein said additional reactant comprises 2,2,2-trifluoroethyl methacrylate.

10. The method of claim 1, wherein said enzyme comprises a lipase.

11. A system, comprising:
a) at least one substrate loading deck for introducing a substrate into a first microchannel;
b) a plurality of reactant loading decks into which at least one reactant is introduced into a second microchannel;
c) a mixing area wherein said substrate from said first microchannel and said reactant from said second microchannel intersect, thereby forming a first reaction mixture in a third microchannel;
d) a first reaction chamber comprising a first enzyme in fluidic communication with said third microchannel wherein said first reaction mixture forms a second reaction mixture; and
e) a second reaction chamber comprising a second enzyme in fluidic communication with said first reaction chamber wherein said second reaction mixture forms a polymer.

12. The system of claim 11, wherein said reaction chamber further comprises a microcomb filter for separating said polymer.

12. The system of claim 12, wherein said reaction chamber further comprises at least one cross channel for collecting unreacted sample and unreacted reactant.

13. The system of claim 11, wherein said first enzyme comprises a lipase.

14. The system of claim 11, wherein said second enzyme comprises a horseradish peroxidase.

15. The system of claim 11, wherein said polymer comprises poly L-ascorbyl methyl methacrylate.

16. A device, comprising:
a) a plurality of microchannels arranged in a symmetric branching configuration, wherein said microchannels have an inlet and an outlet;
b) a plurality of loading decks fluidly connected to said microchannel inlet; and
c) a plurality of reaction chambers fluidity connected to said microchannel outlet.

17. The device of claim 16, wherein said reaction chambers further comprise a microcomb filter.

18. The device of claim 16, wherein said loading decks are selected from the group consisting of a substrate loading deck and a reactant loading deck.

19. The device of claim 16, wherein said microchannel outlet further comprises at least one capillary check valve.

20. The device of claim 16, wherein said symmetric microchannel branching configuration creates a plurality of mixing areas, wherein said mixing areas comprise a Y shape.
